(19) United States
(12) Patent Application Publication
(10) Pub. No.: US 2025/0360168 A1
(43) Pub. Date: Nov. 27, 2025

(54) METHODS FOR MAKING HUMAN GROWTH FACTOR CONCENTRATE AND COMPOSITIONS AND USES THEREOF

(71) Applicant: GMS Capital Partners LLC, Washington, DC (US)

(72) Inventors: Gerhard HODLER, Worb (CH); Rana BUSTAMI, Amman (JO)

(21) Appl. No.: 19/177,447

(22) Filed: Apr. 11, 2025

Related U.S. Application Data
(60) Provisional application No. 63/632,902, filed on Apr. 11, 2024.

(51) Int. Cl. A61K 35/19; A61K 38/18; A61K 38/30; A61K 47/10; A61K 47/18; A61K 47/24; A61K 47/26; A61K 47/42; A61P 17/02

(52) U.S. Cl. CPC A61K 35/19; A61K 38/1808; A61K 38/1841; A61K 38/185; A61K 38/1858; A61K 38/1866; A61K 38/30; A61K 47/10; A61K 47/183; A61K 47/24; A61K 47/26; A61K 47/42; A61P 17/02

(57) ABSTRACT

The present disclosure relates to methods for making platelet derived human growth factor concentrate and related therapeutic and pharmaceutical compositions and uses thereof. Methods of use of the therapeutic and pharmaceutical compositions are also provided herein.

### RELATED APPLICATIONS

This application claims the priority and benefits of U.S. Provisional Application No. 63/632,902, filed Apr. 11, 2024, the contents of which are incorporated herein in their entirety by reference.

### FIELD

The present disclosure relates to methods for making human growth factor concentrate and compositions comprising human growth factors and uses thereof. The compositions can be used in the field of medicine, such as in wound healing.

### BACKGROUND

Growth factors are a group of naturally occurring glycoproteins or steroid hormones that stimulate cellular growth and activate cellular proliferation and/or differentiation. Many growth factors are quite versatile, stimulating cellular division in numerous different cell types; while others are specific to a particular cell-type. Examples of growth factors include platelet-derived growth factor (PDGF), vascular endothelial growth factor (VEGF), transforming growth factor beta 1 (TGF-β1), epidermal growth factor (EGF), fibroblast growth factor (FGF), and human platelet derived growths factors (PDGF-) AA, AB and BB. Recombinant human PDGF-BB (rhPDGF-BB) is approved in both the United States and Europe for human use in topical applications to accelerate healing of chronic diabetic foot sores.

Platelets are a rich source of growth factors. Fresh platelets have a very short shelf life. Previous methods of isolating growth factors from platelets describe using a single and fresh platelet concentrate suitable for transfusion, which leads to a substantial variability of growth factor concentration. A human growth factor concentrate suitable for pharmaceutical production is needed. Disclosed herein are methods and compositions to address this need.

### SUMMARY

The present disclosure provides improved methods for isolating growth factors from platelets.

In some embodiments, the present disclosure provides methods for making a therapeutic composition comprising one or more human platelet derived growth factors, the methods comprising:
(a) collecting a supernatant from a separated platelet lysate to obtain a solution comprising growth factors; optionally filtering the supernatant to obtain a clear solution;
(b) inactivating viruses in the solution of step (a) by a first virus elimination step; and
(c) generating a filtrate by passing the solution from step (b) through a nanofilter as a second virus elimination step.

In some embodiments, the methods further comprise concentrating the filtrate from step (c) to obtain a suitable concentration of the one or more human platelet derived growth factors.

In some embodiments, the present disclosure provides methods for making a therapeutic composition comprising one or more human platelet derived growth factors, the methods comprising:
(a) providing platelet lysates from at least two donors;
(b) diluting the platelet lysates with a serum-free diluent to create a mixture;
(c) separating the mixture into a supernatant and a pellet;
(d) collecting the supernatant to obtain a solution comprising growth factors; optionally filtering the supernatant to obtain a clear solution;
(e) inactivating viruses in the solution of step (d) by a first virus elimination step;
(f) generating a filtrate by passing the solution from step (e) through a nanofilter as a second virus elimination step; and
(g) concentrating the filtrate from step (f) to obtain a suitable concentration of the one or more human platelet derived growth factors.

In some embodiments of the methods for making a therapeutic composition comprising one or more human platelet derived growth factors of the present disclosure, the platelet lysates are previously frozen.

In some embodiments of the methods for making a therapeutic composition comprising one or more human platelet derived growth factors of the present disclosure, the platelet lysates are obtained from human platelet concentrates, buffy coats, or platelet rich plasma, where obtaining the platelet lysates comprises lysis of at least a portion of the platelets in the platelet concentrates, buffy coats, or platelet rich plasma.

In some embodiments of the methods for making a therapeutic composition comprising one or more human platelet derived growth factors of the present disclosure, the platelet concentrates, buffy coats, or platelet rich plasma are negative for the presence of pathogens.

In some embodiments of the methods for making a therapeutic composition comprising one or more human platelet derived growth factors of the present disclosure, at least a portion of plasma is removed from the platelets and replaced by a serum-free solution prior to the lysis of the platelets.

In some embodiments of the methods for making a therapeutic composition comprising one or more human platelet derived growth factors of the present disclosure, obtaining the platelet lysates comprises:
(a) separating the human platelet concentrates or platelet rich plasma into a supernatant and a pellet containing the platelets;
(b) resuspending the pellet containing the platelets into a solution, where the solution is serum-free and/or comprises human albumin;
(c) freezing the resuspended platelets; and
(d) thawing the resuspended platelets.

In some embodiments of the methods for making a therapeutic composition comprising one or more human platelet derived growth factors of the present disclosure, freezing and thawing of the resuspended platelets are repeated for one or more times.

In some embodiments of the methods for making a therapeutic composition comprising one or more human platelet derived growth factors of the present disclosure, the resuspended platelets reach a core temperature of at least −18° C. after freezing.

In some embodiments of the methods for making a therapeutic composition comprising one or more human platelet derived growth factors of the present disclosure, the platelets are stored at a temperature of at least −18° C., preferably below −30° C., after freezing. In certain embodiments, the platelets are stored for a period of up to 12 months after freezing.

In some embodiments of the methods for making a therapeutic composition comprising one or more human platelet derived growth factors of the present disclosure, the serum-free diluent is a calcium chloride solution.

In some embodiments of the methods for making a therapeutic composition comprising one or more human platelet derived growth factors of the present disclosure, the concentration of the calcium chloride is 15-25 mM. In certain embodiments, the concentration of the calcium chloride is 20 mM.

In some embodiments of the methods for making a therapeutic composition comprising one or more human platelet derived growth factors of the present disclosure, one or more detergents and/or solvents are added to the mixture prior to dilution of the platelet lysates with the serum-free diluent. In some embodiments, the one or more detergents comprises a non-ionic detergent. In some embodiments, the one or more detergents comprises a polysorbate derivative, a nonylphenol derivative, a cholic acid derivative, or a combination thereof.

In some embodiments of the methods for making a therapeutic composition comprising one or more human platelet derived growth factors of the present disclosure, the one or more detergents added to the mixture comprises a polysorbate derivative. In certain embodiments, the polysorbate derivative is polysorbate 80 (Tween 80).

In some embodiments of the methods for making a therapeutic composition comprising one or more human platelet derived growth factors of the present disclosure, the one or more detergents added to the mixture comprises a nonylphenol derivative. In certain embodiments, the nonylphenol derivative is Triton-X-100.

In some embodiments of the methods for making a therapeutic composition comprising one or more human platelet derived growth factors of the present disclosure, the one or more detergents added to the mixture comprises a cholic acid derivative. In certain embodiments, the cholic acid derivative is sodium cholate.

In some embodiments of the methods for making a therapeutic composition comprising one or more human platelet derived growth factors of the present disclosure, the concentration of the detergent in the mixture is 1%.

In some embodiments of the methods for making a therapeutic composition comprising one or more human platelet derived growth factors of the present disclosure, the one or more solvents added to the mixture comprises tri-n-butyl phosphate. In certain embodiments, the concentration of the solvent in the mixture is 0.3%.

In some embodiments of the methods for making a therapeutic composition comprising one or more human platelet derived growth factors of the present disclosure, the first virus elimination step comprises heating the solution, lowering the pH of the solution, exposing the solution to UV light, nanofiltration of the solution, or adding solvent/detergent reagents to the solution.

In some embodiments of the methods for making a therapeutic composition comprising one or more human platelet derived growth factors of the present disclosure, the first virus elimination step comprises exposing the solution to UV light.

In some embodiments of the methods for making a therapeutic composition comprising one or more human platelet derived growth factors of the present disclosure, the first virus elimination step comprises heating the solution. In some embodiments, the solution is heated to about 60° C. In certain embodiments, the solution is heated to said temperature for at least two hours.

In some embodiments of the methods for making a therapeutic composition comprising one or more human platelet derived growth factors of the present disclosure, the first virus elimination step comprises lowering the pH of the solution. In some embodiments, the pH of the solution is lowered to about 4.0. In certain embodiments, the solution is incubated at said pH for up to 10 hours. In some embodiments, the solution is incubated at about 37° C.

In some embodiments of the methods for making a therapeutic composition comprising one or more human platelet derived growth factors of the present disclosure, the first virus elimination step comprises adding solvent/detergent reagents to the solution.

In some embodiments of the methods for making a therapeutic composition comprising one or more human platelet derived growth factors of the present disclosure, the solvent/detergent reagents added to the solution comprise a non-ionic detergent.

In some embodiments of the methods for making a therapeutic composition comprising one or more human platelet derived growth factors of the present disclosure, the solvent/detergent reagents added to the solution comprise a polysorbate derivative, a nonylphenol derivative, a cholic acid derivative, or a combination thereof.

In some embodiments of the methods for making a therapeutic composition comprising one or more human platelet derived growth factors of the present disclosure, the solvent/detergent reagents added to the solution comprise a polysorbate derivative. In certain embodiments, the polysorbate derivative is polysorbate 80 (Tween 80).

In some embodiments of the methods for making a therapeutic composition comprising one or more human platelet derived growth factors of the present disclosure, the solvent/detergent reagents added to the solution comprise a nonylphenol derivative. In certain embodiments, the nonylphenol derivative is Triton-X-100.

In some embodiments of the methods for making a therapeutic composition comprising one or more human platelet derived growth factors of the present disclosure, the concentration of the detergent in the solution is 1%.

In some embodiments of the methods for making a therapeutic composition comprising one or more human platelet derived growth factors of the present disclosure, the solvent/detergent reagents added to the solution, the one or more solvents added to the solution comprises tri-n-butyl phosphate.

In some embodiments of the methods for making a therapeutic composition comprising one or more human platelet derived growth factors of the present disclosure, the concentration of the solvent in the solution is 0.3%.

In some embodiments of the methods for making a therapeutic composition comprising one or more human platelet derived growth factors of the present disclosure, the solution is passed through a nanofilter with a pore size of 15-25 nm.

In some embodiments of the methods for making a therapeutic composition comprising one or more human platelet derived growth factors of the present disclosure, where a filtrate is generated by passing the solution through a nanofilter and then concentrated, concentrating the filtrate comprises ultrafiltration. In some embodiments, the ultrafiltration is tangential flow filtration. In certain embodiments, the tangential flow filtration is done against 3 to 5 volumes of purified water or a buffer suitable for injection. In certain embodiments, the buffer is saline.

The present disclosure further provides pharmaceutical compositions comprising the therapeutic compositions made by the methods described herein and a pharmaceutically acceptable excipient.

In some embodiments of the pharmaceutical compositions of the present disclosure, the pharmaceutical compositions comprise one or more human platelet derived growth factors selected from the group consisting of: platelet derived growth factor BB (PDGF-BB), epidermal growth factor (EGF), vascular endothelial growth factor (VEGF), nerve growth factor beta (NGFO), Transforming growth factor beta 1 (TGF-β1), platelet derived growth factor AB (PDGF-AB), and insulin-like growth factor 1 (IGF-1). In certain embodiments, the concentration of the one or more human platelet derived growth factors is between 1 and 1000 ng/mL.

In some embodiments of the pharmaceutical compositions of the present disclosure, the pharmaceutical compositions comprise one or more stabilizers selected from the group consisting of human albumin, sucrose, trehalose, maltose, sorbitol, and hydroxyethyl-starch.

In some embodiments of the pharmaceutical compositions of the present disclosure, the pharmaceutical compositions comprise 10% sucrose or trehalose.

In some embodiments of the pharmaceutical compositions of the present disclosure, the pharmaceutical compositions comprise one or more amino acids selected from the group consisting of L-arginine, L-lysine, and N-acetyltryptophan. In certain embodiments, the concentration of the amino acids is 250 mmol/l.

In some embodiments of the pharmaceutical compositions of the present disclosure, the pharmaceutical compositions comprise between 10 and 100 mg/mL of human albumin.

In some embodiments of the pharmaceutical compositions of the present disclosure, the pharmaceutical compositions comprise a buffering agent selected from the group consisting of sodium citrate, sodium phosphate, and sodium acetate. In certain embodiments, the buffering agent maintains a pH of between 6.0 and 8.5, preferably a pH of 7.2.

In some embodiments of the pharmaceutical compositions of the present disclosure, the osmolality of the pharmaceutical compositions is 350 mOsmol/kg or less. In certain embodiments, the osmolality is 300 mOsmol/kg.

In some embodiments of the pharmaceutical compositions of the present disclosure, the pharmaceutical compositions are lyophilized.

In some embodiments of the pharmaceutical compositions of the present disclosure, the pharmaceutical compositions are endotoxin free.

In some embodiments of the pharmaceutical compositions of the present disclosure, the pharmaceutical compositions are free of viruses.

In some embodiments of the pharmaceutical compositions of the present disclosure, the pharmaceutical compositions are free of bloodborne non-enveloped and enveloped viruses.

In some embodiments of the pharmaceutical compositions of the present disclosure, the pharmaceutical compositions are safe for subcutaneous injection, intradermal injection, transdermal injection, subdermal injection, intramuscular injection, and intraarticular injection.

The present disclosure further provides methods of treating a condition or disease in a subject in need thereof, comprising administering to the subject the pharmaceutical compositions of the present disclosure.

In some embodiments of the methods of treating a condition or disease in a subject in need thereof of the present disclosure, the pharmaceutical composition is administered through subcutaneous injection, intradermal injection, transdermal injection, subdermal injection, intramuscular injection, intraarticular injection, topical application, or a combination thereof.

In some embodiments of the methods of treating a condition or disease in a subject in need thereof of the present disclosure, the pharmaceutical composition is administered through subcutaneous injection, intradermal injection, transdermal injection, or subdermal injection.

In some embodiments of the methods of treating a condition or disease in a subject in need thereof of the present disclosure, the condition or disease is selected from the group consisting of non-healing wounds or ulcers, chronic and acute dermatological disorders, intra-abdominal abscesses, neurodegenerative diseases, muscle injuries, tendonitis, ligament injuries, and osteoarthritis. In some embodiments, the non-healing wound or ulcer is selected from the group consisting of diabetic foot ulcers, corneal ulcers, acute wounds, burns, acute external surgical wounds to the epidermis, acute surgical wounds to an internal organ, traumatic wounds, and atraumatic wounds. In some embodiments, the chronic and acute dermatological disorder is eczema or alopecia.

### BRIEF DESCRIPTION OF THE DRAWINGS

FIG. 1 provides a log scale bar chart showing the concentrations of four platelet derived growth factors (PDGF-AB, PDGF-BB, TGF-β1, and EGF) in various resuspension media, including defibrinated plasma, cryo-poor plasma, 3% human albumin solution, and saline.

FIG. 2 provides four individual bar charts showing the concentrations of PDGF-AB, PDGF-BB, TGF-β1, and EGF in various resuspension media, including defibrinated plasma, cryo-poor plasma, 3% human albumin solution, and saline.

FIG. 3 provides a flow chart showing an exemplary process used to prepare human platelet-derived growth factor concentrate (GFC-01).

FIGS. 4A-4E provide line graphs showing the cell viability of human fibroblast, human keratinocytes, rat fibroblasts, and keratinocytes treated with various concentrations of human platelet-derived growth factor concentrate, referred to as GFC-01, and human and rat EGF and TGF-0.

FIG. 5A provides images and FIG. 5B provides a line graph quantifying wound area in a migration assay with human fibroblasts treated with various concentrations of human growth factor concentrate (GFC-01) at indicated time points.

FIG. 6A provides images and FIG. 6B provides a line graph showing wound area in a migration assay with rat fibroblasts treated with various concentrations of human growth factor concentrate (GFC-01) at indicated time points.

FIGS. 7A-7E provides lines graphs showing average wound area in nondiabetic rats (FIG. 7A), control diabetic rats (FIG. 7B), diabetic rats treated with 0.5 ml of GFC-01 (FIG. 7C), diabetic rats treated with 1.0 ml of GFC-01 (FIG. 7D), and diabetic rats treated with 1.5 ml of GFC-01 (FIG. 7E).

### DETAILED DESCRIPTION

### Overview

The present disclosure relates to methods for preparing platelet derived human growth factor concentrate and related therapeutic and pharmaceutical compositions and uses thereof. In some embodiments, the methods disclosed herein are used to isolate and/or concentrate growth factors derived from a starting material comprising platelets, such as human platelet concentrates (PCs), buffy coats (BC), or platelet rich plasma (PRP). In some embodiments, the methods disclosed herein make use of platelets pooled from more than one donor, thus providing a therapeutic composition with reduced variations in growth factor concentrations compared to other methods of preparation. Without being held to theory, pooling platelet concentrates from many donors may level out individual variations and allow for industrial scale pharmaceutical manufacturing of platelet-derived growth factor concentrates. Additionally, fresh platelets suitable for transfusion expire after about 5-7 days and are in short supply. The methods of the present disclosure utilize either fresh platelets or platelets expired for transfusion purposes. In some embodiments, the methods described herein utilize platelets expired for transfusion purposes for the preparation of platelet-derived growth factor concentrates. In some embodiments, the methods described herein are compatible with isolation of growth factors from platelets expired for transfusion purposes. In some embodiments, use of expired platelets extends the storage period and bypasses the shortage of fresh platelets in transfusion medicine. In some embodiments, the methods described herein are also advantageous for industrial scale manufacturing. In some embodiments, the methods of the present disclosure are used for the production of a standardized and safe human growth factor concentrate. In certain embodiments, the human growth factor concentrate generated by said methods meets the criteria for a biological drug product suitable for the intracutaneous injection.

In some embodiments, a method for making a therapeutic composition comprising one or more human platelet derived growth factors comprises:
a. collecting a supernatant from a separated platelet lysate to obtain a solution comprising growth factors; optionally filtering the supernatant to obtain a clear solution;
b. inactivating viruses in the solution of step (a) by a first virus elimination step; and
c. generating a filtrate by passing the solution from step (b) through a nanofilter as a second virus elimination step.

In some embodiments, a method for making a therapeutic composition comprising one or more human platelet derived growth factors comprises:
a. collecting a supernatant from a separated platelet lysate to obtain a solution comprising growth factors; optionally filtering the supernatant to obtain a clear solution;
b. inactivating viruses in the solution of step (a) by a first virus elimination step; and
c. generating a filtrate by passing the solution from step (b) through a nanofilter as a second virus elimination step; and
d. concentrating the filtrate from step (c) to obtain a suitable concentration of the one or more human platelet derived growth factors.

In some embodiments, a method for making a therapeutic composition comprising one or more human platelet derived growth factors comprises:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. nanofiltering the solution from step (e) by passing the solution through a nanofilter, thereby generating a filtrate; and
g. concentrating or diluting the filtrate from step (f) to obtain a suitable concentration of the one or more human platelet derived growth factors.

In some embodiments, a method for making a therapeutic composition comprising one or more human platelet derived growth factors comprises:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter as a second virus elimination step; and
g. concentrating or diluting the filtrate from step (f) to obtain a suitable concentration of the one or more human platelet derived growth factors.

In some embodiments, a method for making a therapeutic composition comprising one or more human platelet derived growth factors comprises:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter, wherein nanofiltering serves as a second virus elimination step; and
g. concentrating or diluting the filtrate from step (f) to obtain a suitable concentration of the one or more human platelet derived growth factors.

While centrifugation is used as an example method for separating suspensions or solutions into a liquid phase and solid phase throughout the methods described herein, it can be appreciated centrifugation may be optional or may be replaced by other methods known in the arts for isolating liquids from solids, such as filtration and precipitation.

### Starting Materials

In one aspect, the methods disclosed herein are used to isolate and/or concentrate growth factors derived from a starting material comprising platelets. In some embodiments, the starting material is human PCs, buffy coats (BC), or platelet rich plasma (PRP). In some embodiments, the PCs, BC, or PRP is collected from healthy donors. For example, human PCs, BC, or PRP can be collected from healthy donors and prepared by a transfusion center that is licensed by a competent authority. Non-limiting examples of methods of preparation of PCs, BC, or PRP in a licensed transfusion center include partial replacement of the plasma by an aqueous solution of a mixture of organic and inorganic sodium salts such as phosphate, citrate, or acetate. One example is the use of InterSol Solution with the Amicus Separator System. InterSol solution is an isotonic solution designed to replace a proportion of the plasma and used as a storage solution for platelet concentrates. The solution contains sodium acetate as a nutrient, sodium citrate to prevent platelet clumping and activation, sodium phosphate for buffering, and sodium chloride for osmolality. In some embodiments, there is partial replacement of plasma with InterSol in preparation of PCs, BC, or PRP. In some embodiments, the PCs, buffy coats, or platelet rich plasma is expired for transfusion.

In some embodiments, the PCs, BC, or PRP are negative for the presence of pathogens according to the current standards in the country of origin of the PCs. In some embodiments, the PCs, BC, or PRP are tested for HIV-1/2 antibodies, HTLV 1/II antibodies, Hepatitis B surface antigen (HBsAG), HCV antibodies, syphilis serology, HIV I RNA, HBV DNA, HCV RNA, West Nile Virus, Chagas, Hepatitis A (HAV), and/or Parvovirus B19. FDA licensed tests, other commercially available tests, or methods known in the art may be used to test for the presence of said pathogens. In some embodiments, the HIV-1/2 antibodies, HTLV 1/II antibodies, Hepatitis B surface antigen (HBsAG), HCV antibodies, syphilis serology, HIV I RNA, HBV DNA, HCV RNA, West Nile Virus, HAV, Parvovirus B19 and/or Chagas are below the detection limit of the assay or test used to check for the presence of said pathogen. In some embodiments, said pathogen is present at less than 10⁴ IU/mL.

### Storable Intermediate of Human Platelets

In some embodiments, individual samples of human PCs, buffy coats (BC), or platelet rich plasma (PRP) are gently centrifuged to avoid lysis of the platelets. The centrifugation can be performed in a collection bag or any suitable container at 100 to 1500 xg for 3 to 20 min. In some embodiments, the human PCs or platelet rich plasma is centrifuged at about 100, 200, 300, 400, 500, 600, 700, 800, 900, 1000, 1100, 1200, 1300, 1400, or 1500 xg. In some embodiments, the human PCs or platelet rich plasma is centrifuged for about 3, 5, 10, 15, or 20 minutes. After centrifugation, the plasmatic supernatant is removed, and the pellet containing the platelets is resuspended in a serum-free solution. In some embodiments, the pellet is resuspended in a protein-free saline solution. In some embodiments, the pellet is resuspended in phosphate buffered saline (PBS). In some embodiments, the pellet is resuspended in saline (0.9% NaCl). In some embodiments, the platelet-containing pellet is resuspended in a human albumin solution. In some embodiments, the pellet is resuspended in a solution comprising 3% human albumin.

In some embodiments, the resuspended platelets are then frozen. In some embodiments, the resuspended platelets are frozen individually. In some embodiments, the resuspended platelets from more than one donor are pooled and frozen as a mini-pool of several PCs. In some embodiments, the resuspended platelets are frozen such that the core temperature reaches at least minus 18° C., preferably at least minus 30° C., for several hours. In some embodiments, the core temperature reaches at least minus 18° C. for 3 hours, 4 hours, 5 hours, 8 hours, 12 hours, or 24 hours. In some embodiments, the core temperature reaches at least minus 18° C. for 12 hours. The frozen resuspended platelets, also referred to as an intermediate, can be immediately processed or stored for up to 12 months at or below minus 18° C. In some embodiments, the intermediate is processed immediately. In some embodiments, the intermediate is stored for more than 1 month, 2 months, 3 months, 4 months, 5 months, 6 months, 7 months, 8 months, 9 months, 10 months, 11 months, or 12 months at or below minus 18° C. In some embodiments, the intermediate is stored for more than 1 month at or below minus 18° C. In some embodiments, the intermediate is stored for more than 3 months at or below minus 18° C. In some embodiments, the intermediate is stored for more than 6 months at or below minus 18° C. In some embodiments, the intermediate is stored for more than 12 months at or below minus 18° C.

### Lysing Platelets

In some embodiments, lysis of platelets releases growth factors. Freeze-thaw cycles can contribute to platelet lysis. In some embodiments, a platelet lysate is obtained by freezing and thawing platelets or an intermediate containing platelets. In some embodiments, a frozen intermediate containing platelets is gently thawed and mixed. In some embodiments, the frozen intermediate is thawed at a temperature of between 2 and 25° C. In some embodiments, several frozen intermediates are thawed. In some embodiments, the intermediate is frozen and thawed again. In some embodiments, the intermediate is frozen and thawed again for one or more times. In some embodiments, the intermediate is frozen and thawed again for two times, three times, four times, or five times. In some embodiments, additional freeze thaw cycles increase the degree of lysis of the platelets. In some embodiments, multiple samples of thawed intermediates can be pooled to obtain a pooled human platelet lysate (hPL) from more than one donor. In some embodiments, the pooled intermediates are frozen and thawed again. In some embodiments, the pooled hPL is frozen and thawed again for one or more times. In some embodiments, the pooled hPL is frozen and thawed again for two times, three times, four times, or five times.

In some embodiments, pooled platelet lysates are derived from at least 10, at least 50, at least 100, at least 500, or at least 1000 donors. In some embodiments, pooled human platelet lysates are derived from at least 10 donors. In some embodiments, pooled human platelet lysates are derived from at least 50 donors. In some embodiments, the pooled human platelet lysates are derived from at least 100 donors. In some embodiments, the pooled human platelet lysates are derived from at least 150 donors. In some embodiments, the pooled human platelet lysates are derived from 50 to 150 donors. In some embodiments, the pooled human platelet lysates are derived from 100 to 150 donors. In some embodiments, the pooled human platelet lysates are derived from 100 to 200 donors. In some embodiments, the pooled human platelet lysates are derived from 100 to 500 donors. In some embodiments, the pooled human platelet lysates are derived from 500 to 1000 donors.

### Isolation of Human Platelet-Derived Growth Factors

A serum-free diluent is then added to the pooled hPL. In some embodiments, the serum-free diluent is a calcium chloride (CaCl₂) solution. In some embodiments, the final concentration of CaCl₂ is about 20 mM. In some embodiments, the serum-free diluent (e.g. CaCl₂ solution) and pooled hPL suspension is incubated for several hours. In some embodiments, the suspension is incubated for at least 3 hours, at least 4 hours, or at least 5 hours. In some embodiments, the suspension is incubated for about 5 hours. In some embodiments, the incubation occurs at a temperature between 2° C. and 25° C. In some embodiments, the incubation occurs at a temperature between 2° C. and 8° C. In some embodiments, the incubation occurs at a temperature between 15° C. and 25° C. In some embodiments, the incubation occurs at a temperature of about 20° C. In some embodiments, the incubation occurs at a temperature of about 22° C. In some embodiments, the incubation activates residual clottable protein in the suspension.

To isolate platelet-derived growth factors, the formation of fibrin clots during processing or application of the preparation needs to be mitigated. Some mitigation methods include elimination of fibrinogen through polyethylene-glycol (PEG) precipitation or use of heparin or other anti-coagulation agents. The methods of the present disclosure do not require a presence of plasma or serum. In some embodiments, residual plasma or serum inherent of the collection of platelets from blood is removed mechanically prior to lysis of platelets. Therefore, control of clot formation is unnecessary. In some embodiments, the methods described herein use a serum-free diluent (e.g. saline or human albumin solution), in place of plasma or serum, to resuspend the platelets before lysis. Without being held to theory or mechanism, in some embodiments, resuspension of platelets in a serum-free diluent (e.g. saline or human albumin solution) dilutes the clottable plasma proteins which remain in the supernatant, which are removed from the platelet containing pellet after centrifugation. In some embodiments, to eliminate any clot forming activity in the downstream processing or during application, remaining clot formation activity, if any, is provoked by incubation of the solution in the presence of CaCl₂, preferably at refrigerated temperatures (e.g. between 2° C. to 8° C.). In some embodiments, the methods of the present disclosure do not require defibrination.

Solvent/detergent (S/D) reagents can also be used to lyse platelets. Addition of S/D reagents may replace one or more freeze/thaw cycles while maintaining sufficient lysis of the platelets. In some embodiments, solvent/detergent reagents are added to lyse platelets further prior to incubation of the pooled suspension with a serum-free diluent (e.g. CaCl₂ solution). In some embodiments, the solvent is tri-n-butylphosphate. In some embodiments, the detergent is a non-ionic detergent, or a combination of several non-ionic detergents. In some embodiments, the detergent is a polysorbate derivative, a nonylphenol derivative, a cholic acid derivative, or a combination thereof. In some embodiments, the detergent comprises a polysorbate derivative. In some embodiments, the detergent comprises polysorbate 80 (Tween 80). In some embodiments, the detergent comprises a nonylphenol derivative. In some embodiments, the detergent comprises Triton-X-100. In some embodiments the detergent comprises sodium cholate. In some embodiments, the detergent comprises Tween 80 and Triton-X-100. In some embodiments the detergent comprises Tween 80 and sodium cholate. In some embodiments, the detergent comprises Triton-X-100 and sodium cholate. In some embodiments, the concentration of the detergent is between 0.1% and 2%. In some embodiments, the final concentration of the detergent in the suspension is 1%. In some embodiments, the final concentration of the solvent in the suspension is between 0.1% and 1%. In some embodiments, the final concentration of the solvent in the suspension is 0.3%. In some embodiments, the solvent/detergent reagents are polysorbate 80 and tri-n-butylphosphate. In some embodiments, the solvent/detergent reagents are Triton-X-100 and tri-n-butylphosphate. In some embodiments, the solvent/detergent reagents are 1% polysorbate 80 and 0.3% of tri-n-butylphosphate.

In some embodiments, the pooled suspension is then separated into a liquid phase (e.g. a supernatant) and a solid phase (e.g. pellet), also referred to as a separate platelet lysate. In some embodiments, the suspension is sharply centrifuged to pellet cell debris and clotted protein, if any. For example, the suspension can be centrifuged at 3000 to 4000×g for 15 to 60 minutes. In some embodiments, the suspension is centrifuged at 3000×g. In some embodiments, the suspension is centrifuged for 4000×g. In some embodiments, the suspension is centrifuged for 15, 20, 30, 45, or 60 minutes. The supernatant from a separated platelet lysate is collected to obtain a solution which contains human growth factors. In some embodiments, the solution is filtered to obtain a clear solution containing the growth factors. In some embodiments, the solution is filtered more than once to obtain a clear solution containing the growth factors. In some embodiments, the filter is between 0.1 μm and 1 μm in size.

### Virus Elimination

In some embodiments, the solution containing human growth factors is then subjected to a first virus elimination step and a second virus elimination step. Non-limiting examples of a virus elimination step include treatment with heat, treatment with low-pH, treatment with solvent/detergent reagents, treatment with ultraviolet (UV) light, nanofiltration, or a combination thereof. In some embodiments, a virus elimination step comprises treatment with heat, treatment with low-pH, treatment with solvent/detergent reagents, treatment with ultraviolet (UV) light, or nanofiltration.

In some embodiments, the virus elimination step comprises heat treatment. In some embodiments, heat treatment comprises heating the solution to about 60° C. In some embodiments, the first virus elimination step comprises treatment with heat. In some embodiments, the second virus elimination step comprises treatment with heat. In some embodiments, the solution is heated for at least 1 hour. In some embodiments, the solution is heated for 1 hour, 2 hours, 3 hours, 4 hours, or 5 hours. In some embodiments, the solution containing human growth factors is heated to about 60° C. for about 3 hours. The heat-treated solution is then cooled. In some embodiments, the heat-treated solution is cooled to between 2 and 25° C. In some embodiments, the solution is cooled to between 2° C. and 8° C. In some embodiments, the solution is cooled to between 15° C. and 25° C. In some embodiments, the solution is cooled to about 4° C. In some embodiments, the solution is cooled to about 20° C.

In some embodiments, the virus elimination step comprises treatment with low-pH. In some embodiments, the first virus elimination step comprises treatment with low-pH. In some embodiments, the second virus elimination step comprises treatment with low-pH. In some embodiments, treatment with low-pH comprises adjusting the solution containing human growth factors to a pH of about 4.0. In some embodiments, an acid is added to the solution to adjust the pH to about 4.0. In some embodiments, the acid is HCl. In some embodiments, the solution is incubated at low-pH for up to 10 hours. In some embodiments, the solution is incubated at low-pH for at least 1 hour. In some embodiments, the solution is incubated at low-pH for 1 hour, 2 hours, 3 hours, 4 hours, 5 hours, 6 hours, 7 hours, 8 hours, 9 hours, or 10 hours. In some embodiments, the incubation occurs at a temperature between 3° and 37° C. In some embodiments, the incubation occurs at 37° C. and the low-pH solution is then cooled. In some embodiments, the low-pH solution is cooled to between 2 and 25° C. In some embodiments, the solution is cooled to between 2° C. and 8° C. In some embodiments, the solution is cooled to between 15° C. and 25° C. In some embodiments, the solution is cooled to about 4° C. In some embodiments, the solution is cooled to about 20° C. In some embodiments the pH is adjusted back to 6.0-7.5 by addition of a neutralizing agent such as NaOH.

In some embodiments, the virus elimination step comprises treatment with solvent/detergent reagents. In some embodiments, the first virus elimination step comprises treatment with solvent/detergent. In some embodiments, the second virus elimination step comprises treatment with solvent/detergent. In some embodiments, the solvent is tri-n-butylphosphate. In some embodiments, the detergent is a non-ionic detergent, or a combination of several non-ionic detergents. In some embodiments, the detergent is a polysorbate derivative, a nonylphenol derivative, a cholic acid derivative, or a combination thereof. In some embodiments, the detergent comprises a polysorbate derivative. In some embodiments, the detergent comprises polysorbate 80 (Tween 80). In some embodiments, the detergent comprises a nonylphenol derivative. In some embodiments, the detergent comprises Triton-X-100. In some embodiments the detergent comprises sodium cholate. In some embodiments, the detergent comprises Tween 80 and Triton-X-100. In some embodiments the detergent comprises Tween 80 and sodium cholate.

In some embodiments, the detergent comprises Triton-X-100 and sodium cholate. In some embodiments, the concentration of the detergent is between 0.1% and 2%. In some embodiments, the final concentration of the detergent in the suspension is 1%. In some embodiments, the final concentration of the solvent in the suspension is between 0.1% and 1%. In some embodiments, the final concentration of the solvent in the suspension is 0.3%. In some embodiments, the solvent/detergent reagents are polysorbate 80 and tri-n-butylphosphate. In some embodiments, the solvent/detergent reagents are Triton-X-100 and tri-n-butylphosphate. In some embodiments, the solvent/detergent reagents are 1% polysorbate 80 and 0.3% of tri-n-butylphosphate.

In some embodiments, the solvent/detergent reagents are added to the CaCl₂ and hPL suspension. In some embodiments, the solvent/detergent reagents are added to the solution which contains the human growth factors, obtained by collecting the supernatant from the hPL suspension as described above. The solution treated with S/D reagents is then incubated. In some embodiments, the solution treated with S/D reagents is incubated at a temperature of at least 30° C. In some embodiments, the incubation occurs at 30° C. In some embodiments, the incubation occurs at 37° C. In some embodiments, the solution treated with S/D reagents is incubated for at least 1 hour. In some embodiments, the solution is incubated for 1 hour, 2 hours, or 3 hours. In some embodiments, the solution treated with S/D reagents is incubated at 30° C. for 1 hour. In some embodiments, the solution is incubated at 37° C. for 1 hour. Optionally, the solution treated with S/D reagents is then cooled. In some embodiments, the solution is cooled to between 2 and 25° C. In some embodiments, the solution is cooled to between 2° C. and 8° C. In some embodiments, the solution is cooled to between 15° C. and 25° C. In some embodiments, the solution is cooled to about 4° C. In some embodiments, the solution is cooled to about 20° C.

The S/D reagents are then removed. In some embodiments, the S/D reagents are removed by soybean oil extraction, hydrophobic interaction chromatography, ultrafiltration or a combination thereof. In some embodiments, the S/D reagents are removed by soybean oil extraction. In some embodiments, the S/D reagents are removed by hydrophobic interaction chromatography. In some embodiments, the S/D reagents are removed by ultrafiltration. In some embodiments, the S/D reagents are removed by soybean oil extraction and hydrophobic interaction chromatography. In some embodiments, the S/D reagents are removed by hydrophobic interaction chromatography and ultrafiltration. In some embodiments, the S/D reagents are removed by soybean oil extraction and ultrafiltration.

In some embodiments, the virus elimination step comprises UV light treatment. In some embodiments, the first virus elimination step comprises UV light treatment. In some embodiments, the second virus elimination step comprises UV light treatment. In some embodiments, UV light treatment comprises exposure to UV-A, UV-B, or UV-C light. In certain embodiments, treatment comprises exposure to UV-A light. In certain embodiments, exposure to UV light is done in the presence of one or more psoralen compounds. In some embodiments, the one or more psoralen compounds comprises aminomethyltrimethylpsoralen (AMT), amotosalen, or a combination thereof. In some embodiments, UV light treatment comprises exposure to UV-A light in the presence of AMT. In some embodiments, UV light treatment comprises exposure to UV-A light in the presence of amotosalen.

In some embodiments, the virus elimination step comprises nanofiltration. In some embodiments, the first virus elimination step comprises nanofiltration. In some embodiments, the second virus elimination step comprises nanofiltration. In some embodiments, the nanofilter has a pore size of between 15 and 25 nm. In some embodiments, the nanofilter has a pore size of 20 nm.

In some embodiments, after treatment with heat, low-pH, and/or S/D reagents and after the solution is cooled as described above, the solution is centrifuged to remove particles formed during heating, if any. In some embodiments, the solution is centrifuged and/or filtered. The supernatant or filtrate is collected to obtain a clarified solution. The clarified solution is then filtered through a nanofilter. In some embodiments, filtration through a nanofilter serves as a second virus elimination step as described above. Virus elimination, such as UV light treatment and nanofiltration, can be applied at any step during processing.

Using the methods described herein, a final solution is obtained which comprises the target proteins of human growth factors that have been separated from the platelets and which has undergone at least two virus elimination steps. The final solution is then ultrafiltered to remove processing agents, such as CaCl₂ or phosphate salts. In some embodiments, human albumin is added to the final solution prior to ultrafiltration. In some embodiments, the concentration of the human albumin in the final solution is between 10 mg/L and 100 mg/L. In some embodiments, without being held to theory or mechanism, the presence of human albumin reduces the loss of the target proteins during ultrafiltration. In some embodiments, the ultrafiltration is done by tangential flow filtration. In some embodiments, the tangential flow filtration is performed against 3 to 5 volumes of purified water. In some embodiments, the tangential flow filtration is performed against 3 to 5 volumes of saline. In certain embodiments, the saline is normal saline (0.9% NaCl). In some embodiments, the ultrafiltration concentrates the solution to a desired concentration of growth factors. In some embodiments, the ultrafiltration concentrates the growth factors to between 1 and 1000 ng/ml. In some embodiments, the ultrafiltration concentrates the growth factors to between 1 and 100 ng/ml. In some embodiments, the ultrafiltration concentrates the growth factors to between 10 and 1000 ng/ml. In some embodiments, the ultrafiltration concentrates the growth factors to between 100 and 500 ng/ml.

### Formulation

In some embodiments, after ultrafiltration, the concentration of one or more target proteins in the solution containing the concentrated platelet derived growth factors is determined by ELISA. In some embodiments, the solution containing the concentrated platelet derived growth factors comprises one or more growth factors selected from the group consisting of platelet derived growth factor AB (PDGF-AB), growth factor BB (PDGF-BB), epidermal growth factor (EGF), vascular endothelial growth factor (VEGF), nerve growth factor beta (NGFO), transforming growth factor beta 1 (TGF-β1), insulin-like growth factor 1 (IGF-1), insulin-like growth factor 2 (IGF-2), VEGF-A, Bone Morphogenetic Protein 4 (BMP-4), fibroblast growth factor 2 (FGF-2), hepatocyte growth factor (HGF), fibronectin, granulocyte colony-stimulating factor (G-CSF), granulocyte-macrophage colony-stimulating factor (GM-CSF), tissue inhibitor of metalloproteinase 1 (TIMP-1), tissue inhibitor of metalloproteinase 4 (TIMP-4), interleukin-6 (IL-6), monocyte chemoattractant protein-1 (MCP-1), stromal cell-derived factor-1 (SDF-1), tumor necrosis factor (TNF), brain-derived neurotrophic factor (BDNF) and a combination thereof. In some embodiments, the solution comprises PDGF-AB. In some embodiments, the solution comprises PDGF-BB. In some embodiments, the solution comprises EGF. In some embodiments, the solution comprises VEGF. In some embodiments, the solution comprises NGFβ. In some embodiments, the solution comprises TGF-1.

In some embodiments, the concentration of TGF-β1 is determined by ELISA. In some embodiments, the concentration of PDGF-AB is determined by ELISA. In some embodiments, the concentration of PDGF-BB is determined by ELISA. In some embodiments, the concentration of EGF is determined by ELISA. In some embodiments, the concentration of VEGF is determined by ELISA. In some embodiments, the concentration of NGFβ is determined by ELISA.

In some embodiments, the solution containing the concentrated platelet derived growth factors is then formulated into a pharmaceutical composition comprising a suitable concentration of growth factors. In some embodiments, the final concentration of the growth factors in the final formulation is between 1 and 1,000 ng/ml. In some embodiments, the final concentration of the growth factors is between 1 and 250 ng/ml. In some embodiments, the final concentration of the growth factors is between 50 and 150 ng/ml. In some embodiments, the final concentration of the growth factors is about 100 ng/ml. In some embodiments, the final concentration of the growth factors is about 50 ng/ml. In some embodiments, the final concentration of PDGF-BB is between 1-80 ng/ml. In some embodiments, the final concentration of PDGF-BB is between 1-40 ng/ml. In some embodiments, the final concentration of EGF is between 0.1 and 25 ng/ml. In some embodiments, the final concentration of EGF is between 0.1 and 10 ng/ml. In some embodiments, the final concentration of VEGF is between 0.01 and 5 μg/ml. In some embodiments, the final concentration of VEGF is between 0.01 and 1 μg/ml. In some embodiments, the final concentration of TGF-β1 is between 5 and 500 ng/ml. In some embodiments, the final concentration of TGF-β1 is between 100 and 400 ng/ml. In some embodiments, the final concentration of TGF-β1 is between 200 and 400 ng/ml. In some embodiments, the final concentration of TGF-β1 is between 240 and 360 ng/ml. In some embodiments, the final concentration of PDGF-AB is between 1 and 100 ng/ml. In some embodiments, the final concentration of PDGF-AB is between 1 and 50 ng/ml.

In some embodiments, the final concentration of total protein in the final formulation is between 10 and 50 g/l. Total protein concentration can be determined by methods known in the art, such as a Bradford assay. In some embodiments, the pH of the final formulation is between 6.5-7.5. In some embodiments, the conductivity of the final formulation is less than 2 mS/cm. In some embodiments, the endotoxin level of the final formulation is less than 0.5 IU/ml. In some embodiments, the albumin concentration of the final formulation is between 10 and 50 g/l. In some embodiments, the albumin concentration of the final formulation is between 24 and 36 g/l.

In some embodiments, the pharmaceutical composition comprising the growth factor concentrate described herein contains one or more stabilizers, amino acids, other pharmaceutically acceptable excipients, or a combination thereof. In some embodiments, the pharmaceutical composition comprises a stabilizer. Examples of stabilizers include, without limitation, human albumin, sucrose, trehalose, maltose, sorbitol, hydroxyethyl-starch, citric acid, ascorbic acid, oleic acid, cape acid, capric acid, polyvinylpyrrolidone, waxes, block co-polymers, poloxamers, Poloxamer 188 and 407, poloxamines, Poloxamine 908, polyvinyl pyrrolidone, polyvinyl alcohol, gelatine, polysaccharide, hyaluronic acid, chitosan, derivatives of chitosan, polyacryl acid, derivatives of polyacryl acid, polycarbophil, cellulose derivatives, methyl cellulose, hydroxypropyl cellulose, carboxymethyl cellulose, sugar esters, saccharose monostearate, sodium citrate individually, fatty acids, fatty alcohols, alcohols, long chain fatty acid esters, long chain ethers, hydrophilic derivatives of fatty acids, polyvinylethers, polyvinyl alcohols, hydrocarbons, hydrophobic polymers, moisture-absorbing polymers, and combinations thereof. In some embodiments, the stabilizer is selected from the group consisting of human albumin, sucrose, trehalose, maltose, sorbitol, and hydroxyethyl-starch. In some embodiments, the stabilizer is a di-saccharide. In some embodiments, the stabilizer is sucrose or trehalose. In some embodiments, the stabilizer is sucrose. In some embodiments, the stabilizer is trehalose. In some embodiments, the concentration of the stabilizer is between 1% and 15%. In some embodiments, the concentration of the stabilizer is about 1%. In some embodiments, the concentration of the stabilizer is about 5%. In some embodiments, the concentration of the stabilizer is about 10%.

In some embodiments, the pharmaceutical composition described herein contains one or more amino acids. In some embodiments, the one or more amino acids is selected from the group consisting of arginine, lysine, tryptophan, or a combination thereof. In some embodiments, the one or more amino acids is L-arginine, L-lysine, N-acetyltryptophan, or a combination thereof. In some embodiments, the one of more amino acids is L-arginine. In some embodiments, the one or more amino acids is L-lysine. In some embodiments, the one or more amino acids is N-acetyltryptophan. In some embodiments, the concentration of the one or more amino acids is between 50 and 350 mmol/l. In some embodiments, the concentration of the one or more amino acids is about 250 mmol/l.

In some embodiments, the pharmaceutical composition contains human albumin. In certain embodiments, the concentration of the human albumin in the final formulation is between 10 and 100 mg/ml. In some embodiments, the concentration of the human albumin is about 30 mg/ml.

In some embodiments, the pH of the pharmaceutical composition is adjusted to between 6.0 and 7.5. In some embodiments, the pH of the pharmaceutical composition is about 6.5. In some embodiments, HCl is added to adjust the pH. In some embodiments, NaOH is added to adjust the pH. In some embodiments, the pharmaceutical composition comprises a buffering agent. In certain embodiments, the buffering agent is selected from the group consisting of sodium citrate, sodium phosphate, and sodium acetate.

In some embodiments, the osmolality of the pharmaceutical composition does not exceed 350 mOsmol/kg. In some embodiments, the osmolality of the pharmaceutical composition is 300 mOsmol/kg.

Exemplary formulations for the pharmaceutical composition of the present disclosure are provided below:
Formulation A. 50-150 ng/ml of human platelet derived growth factors in 10% di-saccharide, such as sucrose or trehalose, with a pH of 6.5
Formulation B. 50-150 ng/ml of human platelet derived growth factors in 250 mmol/1 amino acid, such as L-arginine, L-lysine or N-acetyltryptophan, with a pH of 6.5.
Formulation C. 50-150 ng/ml of human platelet derived growth factors in 5% di-saccharide and 125 mmol/1 amino acid, not exceeding a total osmolality of 350 mOsmol/kg, with a pH of 6.5.
Formulation D. Any one of formulations A, B, or C, and between 10 and 100 mg/ml human albumin, not exceeding a total osmolality of 350 mOsmol/kg.

In some embodiments, the formulated bulk solution is then sterile filtered to obtain a final pharmaceutical composition. In some embodiments, the final pharmaceutical composition is freeze dried or lyophilized. In some embodiments, the final pharmaceutical composition is aliquoted into vials prior to lyophilization.

In some embodiments, a method for making a therapeutic composition comprising one or more human platelet derived growth factors comprises:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter as a second virus elimination step;
g. concentrating or diluting the filtrate from step (f) to obtain a suitable concentration of the one or more human platelet derived growth factors; and
h. formulating the concentrate from step (g) to obtain a pharmaceutical composition suitable for injection.

Also provided herein are therapeutic compositions and pharmaceutical compositions, made by the methods described herein, which meet all required international standards for human blood derived medicinal products. In some embodiments, the compositions of the present disclosure are endotoxin free. In some embodiments, the compositions of the present disclosure are virus-free. In certain embodiments, the compositions are free of enveloped and non-enveloped viruses that are typically transmitted by blood products. In some embodiments, the concentration of an active pharmaceutical ingredient (API) in the compositions is standardized. In some embodiments, the lyophilized compositions of the present disclosure can be stored for up to 24 months when stored at or below 30° C. In some embodiments, a lyophilized pharmaceutical composition needs to be dissolved in water for injection or sterile physiological NaCl solution prior to administration. In some embodiments, the compositions of the present disclosure are sterile and essentially free of endotoxins.

In some embodiments, a therapeutic composition comprising one or more human platelet derived growth factors is prepared by a method comprising:
a. collecting a supernatant from a separate platelet lysate to obtain a solution comprising growth factors; optionally filtering the supernatant to obtain a clear solution;
b. inactivating viruses in the solution of step (d) by a first virus elimination step; and
c. nanofiltering the solution from step (e) by passing the solution through a nanofilter, thereby generating a filtrate.
g. concentrating or diluting the filtrate from step (f) to obtain a suitable concentration of the one or more human platelet derived growth factors.

In some embodiments, a therapeutic composition comprising one or more human platelet derived growth factors is prepared by a method comprising:
a. collecting a supernatant from a separate platelet lysate to obtain a solution comprising growth factors; optionally filtering the supernatant to obtain a clear solution;
b. inactivating viruses in the solution of step (d) by a first virus elimination step;
c. nanofiltering the solution from step (e) by passing the solution through a nanofilter, thereby generating a filtrate; and
d. concentrating or diluting the filtrate from step (f) to obtain a suitable concentration of the one or more human platelet derived growth factors.

In some embodiments, a therapeutic composition comprising one or more human platelet derived growth factors is prepared by a method comprising:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. nanofiltering the solution from step (e) by passing the solution through a nanofilter, thereby generating a filtrate; and
g. concentrating or diluting the filtrate from step (f) to obtain a suitable concentration of the one or more human platelet derived growth factors.

In some embodiments, a therapeutic composition comprising one or more human platelet derived growth factors is prepared by a method comprising:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter as a second virus elimination step; and
g. concentrating or diluting the filtrate from step (f) to obtain a suitable concentration of the one or more human platelet derived growth factors.

In some embodiments, a therapeutic composition comprising one or more human platelet derived growth factors is prepared by a method comprising:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter, wherein nanofiltering serves as a second virus elimination step; and
g. concentrating or diluting the filtrate from step (f) to obtain a suitable concentration of the one or more human platelet derived growth factors.

### Methods of Use

Without being held to theory or mechanism, in some embodiments, the compositions of the present disclosure may aid in tissue regeneration, at least in part, by promoting the growth of tissues. For example, they can directly promote the growth and differentiation of cells to produce such tissues. In some embodiments, the compositions and methods of the present disclosure are used to promote tissue growth and repair and/or enhance healing compared to natural healing or healing supplemented by addition of systemic antibiotics. In some embodiments, when applied to damaged or diseased tissues, the compositions of the present disclosure can lead to restoration of these tissues, thereby improving the prognosis for affected areas.

In some embodiments, a composition described herein is administered to treat a condition or disease in a subject in need thereof. In some embodiments, a composition of the present disclosure is formulated for subcutaneous injection, intradermal injection, transdermal injection, subdermal injection, intramuscular injection, or intraarticular injection. In some embodiments, a composition described herein is administered to a subject by any suitable means including, but are not limited to, subcutaneous injection, intradermal injection, transdermal injection, subdermal injection, intramuscular injection, intraarticular injection, or topical application. In some embodiments, a composition of the present disclosure is administered to a subject by subcutaneous injection, intradermal injection, transdermal injection, subdermal injection, intramuscular injection, intraarticular injection, topical application, or a combination thereof. In some embodiments, a composition described herein is administered through subcutaneous injection, intradermal injection, transdermal injection, or subdermal injection. In certain embodiments, a composition described herein is administered to a subject by subcutaneous injection. In certain embodiments, a composition described herein is administered to a subject by intradermal injection. In certain embodiments, a composition described herein is administered to a subject by transdermal injection. In certain embodiments, a composition described herein is administered to a subject by subdermal injection. In certain embodiments, a composition described herein is administered to a subject by intramuscular injection. In certain embodiments, a composition described herein is administered to a subject by topical application.

The therapeutic compositions and pharmaceutical compositions of the present disclosure can be used to treat conditions or diseases such as non-healing wounds or ulcers, chronic and acute dermatological disorders, intra-abdominal abscesses, neurodegenerative diseases, muscle injuries, and osteoarthritis. Non-limiting examples of non-healing wounds or ulcers include diabetic foot ulcers, corneal ulcers, acute wounds, burns, acute external surgical wounds to the epidermis, acute surgical wounds to an internal organ, traumatic wounds, and atraumatic wounds. Examples of chronic and acute dermatological disorders include eczema and alopecia (hair loss). The therapeutic compositions and pharmaceutical compositions of the present disclosure can be used to treat conditions or diseases such as tendonitis, ligament injuries, and muscle tears.

Provided herein are methods of treating a subject in need thereof, comprising administering to the subject the platelet-derived growth factor concentrates described herein, a therapeutic composition described herein, or a pharmaceutical composition described herein. In some embodiments of the methods of treating a subject in need thereof, a platelet-derived growth factor concentrate, a therapeutic composition, or a pharmaceutical composition described herein is administered one time, two times, three times, four times, or fives times to the subject. In some embodiments, a platelet-derived growth factor concentrate, a therapeutic composition, or a pharmaceutical composition described herein is administered at least two times, at least three times, at least four times, or at least five times to the subject.

In some embodiments, a platelet-derived growth factor concentrate, a therapeutic composition, or a pharmaceutical composition described herein is administered to the subject at least once a week, at least twice a week, or at least three times a week. In some embodiments, a platelet-derived growth factor concentrate, a therapeutic composition, or a pharmaceutical composition described herein is administered to the subject at least once every two weeks or at least once a month.

In some embodiments of the methods of treating a subject in need thereof, the subject has a non-healing wound or ulcer, a chronic or acute dermatological disorder, an intra-abdominal abscess, a neurodegenerative disease, a muscle injury, or osteoarthritis. In some embodiments, the subject has a non-healing wound or ulcer. In certain embodiments, the non-healing wound or ulcer is a diabetic foot ulcer. In certain embodiments, the non-healing wound or ulcer is a corneal ulcer. In certain embodiments, the non-healing wound or ulcer is an acute wound. In certain embodiments, the non-healing wound or ulcer is a burn. In certain embodiments, the non-healing wound or ulcer is an acute external surgical wounds to the epidermis. In certain embodiments, the non-healing wound or ulcer is an acute surgical wound to an internal organ. In certain embodiments, the non-healing wound or ulcer is a traumatic wound. In certain embodiments, the non-healing wound or ulcer is an atraumatic wound. In some embodiments, the subject has a chronic or acute dermatological disorder. In certain embodiments, the chronic or acute dermatological disorder is eczema. In certain embodiments, the chronic or acute dermatological disorder is alopecia (hair loss). In some embodiments, the subject has tendonitis. In some embodiments, the subject has a ligament injury. In some embodiments, the subject has a muscle tear.

### Terms and Concepts

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references

For the purposes of the present disclosure, the following terms are defined below.

The terms “a” and “an” and “the” and similar references used in the context of describing a particular embodiment (especially in the context of certain of the following claims) can be construed to cover both the singular and the plural, unless specifically noted otherwise.

The term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Numerical ranges are provided for certain quantities in the present application. It is to be understood that these ranges comprise all subranges therein. Thus, a range of “100 to 150” or “between 100 and 150” includes all possible ranges therein (e.g., 101-149, 102-148, 103-147, 104-146, 105-145, 106-140, etc.). Furthermore, all values within a given range may be an endpoint for the range encompassed thereby (e.g., the range 100-150 includes the ranges with endpoints such as 120-150, 100-125, etc.).

The term “virus elimination” is used to refer to the reduction or inactivation of bloodborne pathogens in blood products. In certain embodiments, reduction or inactivation of pathogens includes procedures for virus inactivation and removal. The therapeutic compositions and pharmaceutical compositions of the present disclosure are free of bloodborne pathogens, such as bacteria, parasites, and non-enveloped and enveloped viruses.

The term “treatment” or “treating” is used to refer to treatment or treating of a subject indicate any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In some embodiments, “treatment” or “treating” includes a complete remission.

The terms “subject” or a “subject in need thereof” or a “patient” or a “patient in need thereof” include a mammalian subject such as a human subject. In some embodiments, the subject is a human.

The term “statistically significant,” is used to describe a result that was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The term “separated platelet lysate” is used to describe a platelet lysate or pooled platelet lysate that has been separated into a solid residue (such as a precipitate or pellet) and a supernatant (the liquid overlying the solid residue). In some embodiments, centrifugation, filtration, or other methods known in the art are used to separate the supernatant from the solid residue. In preferred embodiments, centrifugation is used to separate supernatant from solid matter.

As used herein, the terms “therapeutically effective amount” or “therapeutic dose” is the amount of an agent needed to elicit the desired biological response following administration.

The term “pharmaceutically acceptable excipient” is intended to denote any material, which is inert in the sense that it substantially does not have a therapeutic and/or prophylactic effect per se. Such an excipient is added with the purpose of making it possible to obtain a pharmaceutical composition having acceptable technical properties. For example, a pharmaceutically acceptable excipient can be selected from an oil, solvent, surfactant, antioxidant, polymer, or mixture thereof.

A pharmaceutically acceptable excipient may be used to adjust the osmolality of the pharmaceutical composition. Examples of regulators for adjusting the osmolality include, without limitation, water-soluble, physiologically tolerated compounds such as inorganic salts, e.g., alkali metal salts, preferably sodium chloride, sugars, e.g. sucrose or dextrose, sugar alcohols, e.g., mannitol, or polyalkylene glycols, e.g., polyethylene glycols. It is also possible to use a mixture of at least two representatives of different classes of regulators or at least two representatives of one class of regulators for adjusting the osmolality.

### ENUMERATED EMBODIMENTS

Provided herein are non-limiting exemplary enumerated embodiments.

Embodiment I-1. A method for preparing a therapeutic composition comprising one or more human platelet derived growth factors, comprising of:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter as a second virus elimination step; and
g. concentrating the filtrate from step (f) to obtain a suitable concentration of the one or more human platelet derived growth factors

Embodiment I-2. The method of Embodiment I-1, wherein the platelet lysates are previously frozen.

Embodiment I-3. The method of Embodiment I-1 or I-2, wherein the platelet lysates from step (a) are obtained from human platelet concentrates, buffy coats, or platelet rich plasma, wherein obtaining the platelet lysates comprises lysis of at least a portion of the platelets in the platelet concentrates, buffy coats, or platelet rich plasma.

Embodiment I-4. The method of Embodiment I-3, wherein the platelet concentrates, buffy coats, or platelet rich plasma are negative for the presence of pathogens.

Embodiment I-5. The method of Embodiment I-3 or Embodiment I-4, wherein, prior to lysis of the platelets, at least a portion of plasma is removed from the platelets and a serum-free solution is added.

Embodiment I-6. The method of any one of Embodiments I-3-I-5, wherein obtaining the platelet lysates comprises:
a. separating the human platelet concentrates or platelet rich plasma into a supernatant and a pellet containing the platelets;
b. resuspending the pellet containing the platelets into a solution, wherein the solution is serum-free and/or comprises human albumin;
c. freezing the resuspended platelets; and
d. thawing the resuspended platelets.

Embodiment I-7. The method of Embodiment I-6, wherein steps (c), freezing the resuspended platelets, and step (d), thawing the resuspended platelets, are repeated for one or more times.

Embodiment I-8. The method of Embodiment I-6 or Embodiment I-7, wherein the resuspended platelets reach a core temperature of at least −18° C. after freezing.

Embodiment I-9. The method of Embodiment I-8, wherein after freezing, the platelets are stored at a temperature of at least −18° C., preferably below −30° C.

Embodiment I-10. The method of any one of Embodiments I-6-I-9, wherein the platelets are stored for a period of up to 12 months after freezing.

Embodiment I-11. The method of any one of Embodiments I-1-I-10, wherein the serum-free diluent is a calcium chloride solution.

Embodiment I-12. The method of Embodiment I-11, wherein the concentration of the calcium chloride is 15-25 mM.

Embodiment I-13. The method of Embodiment I-12, wherein the concentration of the calcium chloride is 20 mM.

Embodiment I-14. The method of any one of Embodiments I-1-I-13, wherein one or more detergents and/or solvents are added to the mixture prior to dilution of the platelet lysates with the serum-free diluent.

Embodiment I-15. The method of Embodiment I-14, wherein the one or more detergents comprises a non-ionic detergent.

Embodiment I-16. The method of Embodiment I-14 or Embodiment I-15, wherein the one or more detergents comprises a polysorbate derivative, a nonylphenol derivative, a cholic acid derivative, or a combination thereof.

Embodiment I-17. The method of Embodiment I-16, wherein the one or more detergents comprises a polysorbate derivative.

Embodiment I-18. The method of Embodiment I-17, wherein the polysorbate derivative is polysorbate 80 (Tween 80).

Embodiment I-19. The method of Embodiment I-16, wherein the one or more detergents comprises a nonylphenol derivative.

Embodiment I-20. The method of Embodiment I-19, wherein the nonylphenol derivative is Triton-X-100.

Embodiment I-21. The method of Embodiment I-16, wherein the one or more detergents comprises a cholic acid derivative.

Embodiment I-22. The method of Embodiment I-21, wherein the cholic acid derivative is sodium cholate.

Embodiment I-23. The method of any one of Embodiments I-14-I-22, wherein the concentration of the detergent in the mixture is 1%.

Embodiment I-24. The method of any one of Embodiments I-14-I-23, wherein the one or more solvents comprises tri-n-butyl phosphate.

Embodiment I-25. The method of any one of Embodiment I-14-I-24, wherein the concentration of the solvent in the mixture is 0.3%.

Embodiment I-26. The method of any one of Embodiments I-1-I-25, wherein the first virus elimination step comprises heating the solution, lowering the pH of the solution, exposing the solution to UV light, ultrafiltration of the solution, or adding solvent/detergent reagents to the solution.

Embodiment I-27. The method of Embodiment I-26, wherein the first virus elimination step comprises exposing the solution to UV light.

Embodiment I-28. The method of Embodiment I-26, wherein the first virus elimination step comprises heating the solution.

Embodiment I-29. The method of Embodiment I-28, wherein the solution is heated to about 60 degrees Celsius.

Embodiment I-30. The method of Embodiment I-29, wherein the solution is heated to said temperature for at least two hours.

Embodiment I-31. The method of Embodiment I-26, wherein the first virus elimination step comprises lowering the pH of the solution.

Embodiment I-32. The method of Embodiment I-31, wherein the pH of the solution is lowered to about 4.0.

Embodiment I-33. The method of Embodiment I-32, wherein the solution is incubated at said pH for up to 10 hours.

Embodiment I-34. The method of Embodiment I-33, wherein the solution is incubated at about 37 degrees Celsius.

Embodiment I-35. The method of Embodiment I-26, wherein the first virus elimination step comprises adding solvent/detergent reagents to the solution.

Embodiment I-36. The method of Embodiment I-35, wherein the solvent/detergent reagents comprise a non-ionic detergent.

Embodiment I-37. The method of Embodiment I-35 or Embodiment I-36, wherein the solvent/detergent reagents comprise a polysorbate derivative, a nonylphenol derivative, a cholic acid derivative, or a combination thereof.

Embodiment I-38. The method of Embodiment I-37, wherein the solvent/detergent reagents comprise a polysorbate derivative.

Embodiment I-39. The method of Embodiment I-38, wherein the polysorbate derivative is polysorbate 80 (Tween 80).

Embodiment I-40. The method of Embodiment I-37, wherein the solvent/detergent reagents comprise a nonylphenol derivative.

Embodiment I-41. The method of Embodiment I-40, wherein the nonylphenol derivative is Triton-X-100.

Embodiment I-42. The method of any one of Embodiments I-35-I-41, wherein the concentration of the detergent in the solution is 1%.

Embodiment I-43. The method of any one of Embodiments I-35-I-42, wherein the one or more solvents comprises tri-n-butyl phosphate.

Embodiment I-44. The method of any one of Embodiment I-35-I-43, wherein the concentration of the solvent is 0.3%.

Embodiment I-45. The method of any one of Embodiments I-1-I-44, wherein the nanofilter has a pore size of 15-25 nm.

Embodiment I-46. The method of any one of Embodiments I-1-I-45, wherein concentrating the filtrate comprises ultrafiltration.

Embodiment I-47. The method of Embodiment I-46, wherein the ultrafiltration is tangential flow filtration.

Embodiment I-48. The method of Embodiment I-47, wherein the tangential flow filtration is done against 3 to 5 volumes of purified water or a buffer suitable for injection.

Embodiment I-49. The method of Embodiment I-48, wherein the buffer is saline.

Embodiment I-50. A composition made by the method of any one of Embodiments I-1-49.

Embodiment I-51. A pharmaceutical composition comprising the composition of Embodiment I-50 and a pharmaceutically acceptable excipient.

Embodiment I-52. The pharmaceutical composition of Embodiment I-51, comprising one or more human platelet derived growth factors selected from the group consisting of: platelet derived growth factor BB (PDGF-BB), epidermal growth factor (EGF), vascular endothelial growth factor (VEGF), nerve growth factor beta (NGFO), Transforming growth factor beta 1 (TGF-β1), platelet derived growth factor AB (PDGF-AB), and insulin-like growth factor 1 (IGF-1).

Embodiment I-53. The pharmaceutical composition of Embodiment I-52, wherein the concentration of the one or more human platelet derived growth factors is between 1 and 1000 ng/mL.

Embodiment I-54. The pharmaceutical composition of any one of Embodiments I-51-53, comprising one or more stabilizers selected from the group consisting of human albumin, sucrose, trehalose, maltose, sorbitol, and hydroxyethyl-starch.

Embodiment I-55. The pharmaceutical composition of Embodiment I-54, comprising 10% sucrose or trehalose.

Embodiment I-56. The pharmaceutical composition of any one of Embodiments I-51-55, comprising one or more amino acids selected from the group consisting of L-arginine, L-lysine, and N-acetyltryptophan.

Embodiment I-57. The pharmaceutical composition of Embodiment I-56, wherein the concentration of the amino acids is 250 mmol/l.

Embodiment I-58. The pharmaceutical composition of any one of Embodiments I-51-57, further comprising between 10 and 100 mg/mL of human albumin.

Embodiment I-59. The pharmaceutical composition of any one of Embodiments I-51-58, comprising a buffering agent selected from the group consisting of sodium citrate, sodium phosphate, and sodium acetate.

Embodiment I-60. The pharmaceutical composition of Embodiment I-59, wherein the buffering agent maintains a pH of between 6.0 and 8.5, preferably wherein the pH is 7.2.

Embodiment I-61. The pharmaceutical composition of any one of Embodiments I-51-60, wherein the osmolality is 350 mOsmol/kg or less.

Embodiment I-62. The pharmaceutical composition of Embodiment I-61, wherein the osmolality is 300 mOsmol/kg.

Embodiment I-63. The pharmaceutical composition of any one of Embodiments I-51-62, wherein the composition is lyophilized.

Embodiment I-64. The pharmaceutical composition of any one of Embodiments I-51-63, wherein the composition is endotoxin free.

Embodiment I-65. The pharmaceutical composition of any one of Embodiments I-51-64, wherein the composition is free of viruses.

Embodiment I-66. The pharmaceutical composition of Embodiment I-65, wherein the composition is free of bloodborne non-enveloped and enveloped viruses.

Embodiment I-67. The pharmaceutical composition of any one of Embodiments I-51-I-66, wherein the pharmaceutical composition is safe for subcutaneous injection, intradermal injection, transdermal injection, subdermal injection, intramuscular injection, and intraarticular injection.

Embodiment I-68. A method of treating a condition or disease in a subject in need thereof, comprising administering to the subject the pharmaceutical composition of any one of Embodiments I-51-I-67.

Embodiment I-69. The method of Embodiment I-68, wherein the pharmaceutical composition is administered through subcutaneous injection, intradermal injection, transdermal injection, subdermal injection, intramuscular injection, intraarticular injection, topical application, or a combination thereof.

Embodiment I-70. The method of Embodiment I-69, wherein the pharmaceutical composition is administered through subcutaneous injection, intradermal injection, transdermal injection, or subdermal injection.

Embodiment I-71. The method of any one of Embodiments I-68-I-70, wherein the condition or disease is selected from the group consisting of non-healing wounds or ulcers, chronic and acute dermatological disorders, intra-abdominal abscesses, neurodegenerative diseases, muscle injuries, tendonitis, ligament injuries, and osteoarthritis.

Embodiment I-72. The method of Embodiment I-71, wherein non-healing wound or ulcer is selected from the group consisting of diabetic foot ulcers, corneal ulcers, acute wounds, burns, acute external surgical wounds to the epidermis, acute surgical wounds to an internal organ, traumatic wounds, and atraumatic wounds.

Embodiment I-73. The method of Embodiment I-72, wherein the chronic and acute dermatological disorder is eczema or alopecia.

### EXAMPLES

### Example 1: Preparation of a Storable Intermediate of Human Platelets

Human platelet concentrates, buffy coats, or platelet rich plasma collected from healthy donors by a transfusion centre that is licensed by the competent authority was used as starting material. Preparation of PC or PRP in the licensed transfusion centre included partial replacement of the plasma by an aqueous solution of a mixture of organic and inorganic sodium salts such as chloride, phosphate, citrate, or acetate, such as InterSol Solution. The InterSol Solution was used with the Amicus Separator System. The starting material was tested negative for the presence of pathogens according to the current standards in the country of origin of the platelets. Individual samples of human platelet concentrate, buffy coats, or platelet rich plasma were gently centrifuged in the collection bag. The plasmatic supernatant was removed, and the pellet containing the platelets was resuspended in either a human albumin containing solution or in a protein-free saline solution, such as phosphate buffered saline (PBS). The resuspended platelets were then frozen individually or as a mini-pool of several platelet concentrates in a way that the core temperature reaches at least −18° C. for several hours. The frozen concentrates, also referred to as an intermediate, were immediately processed. The frozen concentrates can also be stored for up to 12 months at or below −18° C.

### Example 2: Preparation of Pooled Platelet Lysate (Pooled hPL)

The intermediate was gently thawed at a temperature of between 2 and 8° C. and mixed. Optionally, the intermediate could again be frozen and thawed as described above for one or more times to maximize the degree of lysis of the platelets. The thawed platelet lysates were then pooled to obtain pooled platelet lysate (hPL) from a pool size of several dozens or up to several hundreds of donors.

### Example 3: Manufacturing of Human Platelet-Derived Growth Factor Concentrate

A calcium chloride solution was added to the pooled hPL to obtain a concentration of about 20 mM CaCl₂. The suspension was incubated for several hours at a temperature between 2° C. and 25° C. to activate any residual clottable protein. The suspension was then sharply centrifuged to remove cell debris and clotted protein, if any. The supernatant was collected and filtered to obtain a clear solution containing the target proteins. The solution was then heated to about 60° C. for not less than 1 hour as a first dedicated virus elimination step and subsequently cooled to between 2 and 25° C. After cooling, the solution was either centrifuged and/or filtered again to remove particles formed during heat treatment, if any. The clarified solution was then filtered through a nanofilter with a pore size between 15 and 25 nm as the 2^{nd} dedicated virus elimination step.

The nanofiltered solution was then ultrafiltered by tangential flow filtration against 3 to 5 volumes of saline or purified water to remove processing agents such as CaCl₂ or phosphate salts and to concentrate the solution to obtain the desired concentration of growth factors in the final formulation.

Optionally, 10-100 mg/ml human albumin is added to the nanofiltered solution as a protective agent and to avoid losses of the target proteins during the tangential flow filtration.

### Example 4: Solvent/Detergent Reagents Maximize Platelet Lysis and Inactivate Viruses

A calcium chloride solution was added to the pooled hPL to obtain a concentration of about 20 mM CaCl₂. The suspension was incubated for several hours at a temperature between 2° C. and 25° C. to activate any residual clottable protein. The solvent/detergent (S/D) reagents of 1% polysorbate 80 and 0.3% of tri-n-butylphosphate were then added to maximise the lysis of the platelets. The suspension was then sharply centrifuged to remove cell debris and clotted protein, if any. The supernatant was collected and filtered to obtain a clear solution containing the target proteins. The solution was then kept at a temperature of at least 30° C. for 1 hour as a first dedicated virus inactivation step. The S/D reagents were then removed by soybean oil extraction or by hydrophobic interaction chromatography. The clarified solution was then nanofiltered, ultrafiltered, and concentrated as described in Example 3.

### Example 5: Use of Low pH to Inactivates Viruses

A clear solution containing the target proteins was obtained as described in Example 3. The solution was then adjusted to a pH of about 4.0 by the addition of HCl and incubated for at least 1 h at a temperature of between 3° and 37° C. as a dedicated virus inactivation step. The solution was then cooled, clarified, nanofiltered, ultrafiltered, and concentrated, as described in Example 3.

### Example 6: Formulation of the Platelet-Derived Growth Factor Concentrates

The concentration of the growth factors in the solution obtained after tangential flow filtration as described in Examples 3-5 was determined by ELISA. The solution was then formulated to one of the following:
a. 1-250 ng/ml, preferably 50-150 ng/ml, of human platelet derived growth factors,
   10% di-saccharide such as sucrose or trehalose, and
   pH 6.5 by addition of HCl or NaOH
b. 1-250 ng/ml, preferably 50-150 ng/ml, of human platelet derived growth factors,
   250 mmol/l amino acid, such as L-Arginine, L-Lysine or N-Acetyltryptophane, and
   pH 6.5 by addition of HCl or NaOH
c. Any combination of excipients in (a) and (b) not exceeding a total osmolality of 350 mOsmol/kg
d. Any combination of excipients in (a), (b) or (c), with the addition of between 10 and 100 mg/ml of human albumin, not exceeding a total osmolality of 350 mOsmol/kg

This final, formulated bulk solution was then sterile filtered, filled in vials and freeze dried to obtain the final product. Long term stability of all of the formulations was obtained through freeze drying at suitable conditions.

### Example 7: Saline and Human Albumin Solution as a Suspension Media Lead to Improved Recovery of Growth Factors from Platelets

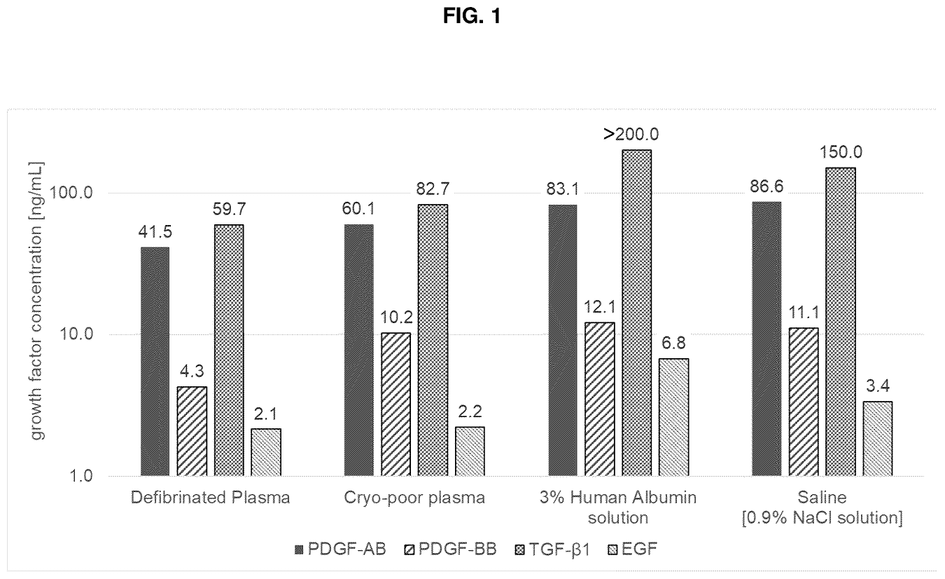

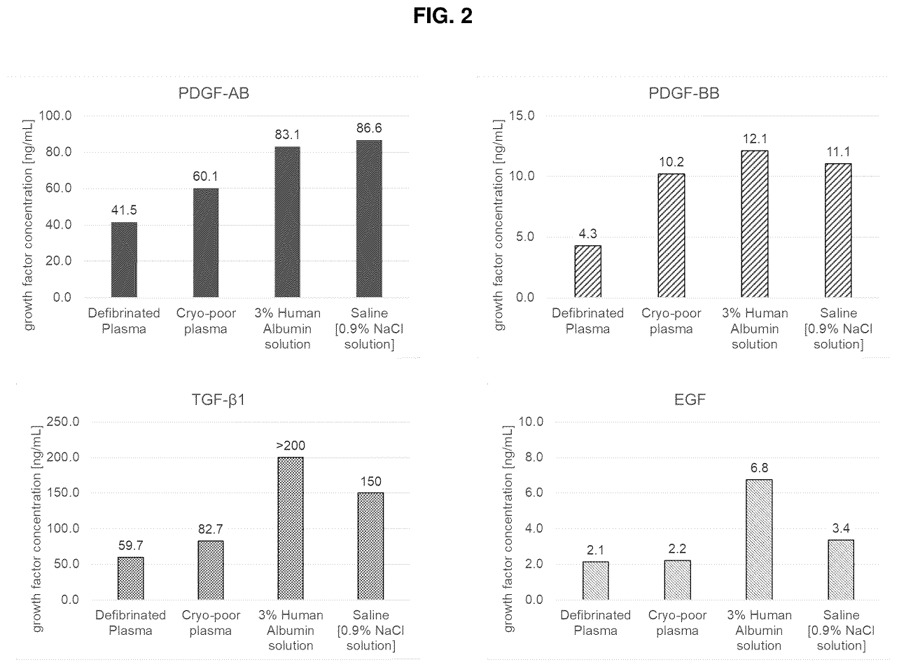

Plasma or serum is commonly used as the resuspension media for platelets. To determine whether other resuspension media can improve the recovery of growth factors from the lysed platelets, various resuspension media were tested in the manufacturing of platelet-derived human growth factor concentrate as described in Example 3. The concentration of four platelet-derived growth factors was determined by ELISA after the growth factors were resuspended in a solution containing 3% human albumin, saline, cryo-poor plasma, or defibrinated plasma. As shown in FIG. 1 and FIG. 2, the concentration of each of platelet derived growth factor AB (PDGF-AB), transforming growth factor beta 1 (TGF-β1), platelet derived growth factor BB (PDGF-BB), and epidermal growth factor (EGF) was surprisingly significantly higher when defibrinated plasma or cryo-poor plasma was replaced by a solution containing 3% human albumin or saline as a resuspension media. Use of the albumin solution and saline yielded a concentration of PDGF-AB that was over 130%, a concentration of TGF-01 that was over 180%, and a concentration of EGF that was 150%-300% compared to concentrations of those obtained with plasma as a resuspension media. Compared to defibrinated plasma, use of the albumin solution and saline gave yields that were about 200% for PDGF-AB, about 250% for PDGF-BB, about 150-300% for EGF and about 250-330% for TGF-β1. These results show that manufacturing platelet-derived human growth factor concentrate using saline or an albumin containing solution as a resuspension media led to improved release of growth factors from the platelets. Therefore, the methods of the present disclosure are more efficient compared to prior methods that use plasma.

### Example 8: Solvent/Detergent Reagents Lead to More Efficient Lysis of Platelets

To determine whether the addition of solvent/detergent reagents can maximize lysis of the platelets and lead to higher recovery of growth factors, pooled hPL are subjected to one to three cycles of freezing and thawing as described in Example 2 with or without the addition of a non-ionic detergent, preferably a polysorbate- or nonylphenol derivative, such as Tween 80 or Triton X 100, respectively, and 0.3% of tri-n-butylphosphate as described in Example 4. The concentrations of each of the growth factors are determined by ELISA and compared between each of the test conditions.

### Example 9: Concentration of Growth Factors in Fresh and Expired Platelet Concentrates

Fresh platelets suitable for transfusion expire after about 5 days and are in short supply. Use of expired platelet concentrates may enhance the availability of platelets as a starting material since these are no longer suitable for transfusion. Growth factors were isolated from fresh platelet concentrates or expired platelet concentrates. The concentration of each of the growth factors is determined by ELISA and compared between the two test conditions.

### Example 10: Example Process for Preparing Human Platelet-Derived Growth Factor Concentrate

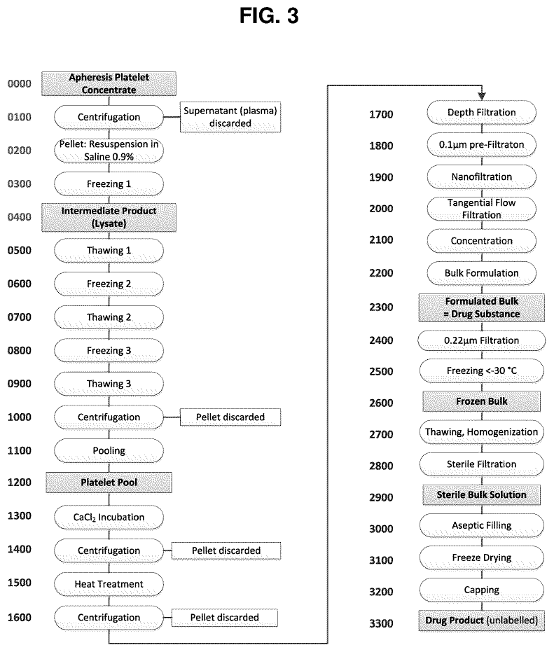

FIG. 3 depicts a flow chart showing the steps in an exemplary process used to prepare human platelet-derived growth factor concentrate. Table 1 below provides descriptions of each step shown in FIG. 3, with each step labeled by a step identifier. It can be appreciated that a step may be optional and that the steps may be performed in a different order to obtain a final product of human platelet-derived growth factor concentrate. Additionally, the conditions described for each step presented in FIG. 3 and Table 1 are provided as an example method by which the step can be performed, but other methods or conditions known in the art may be used to achieve the purpose of a step.

TABLE 1
Example of preparation process for human platelet-derived growth factor concentrate

| Step | | | |
|---|---|---|---|
| identifier | Process Step | Description/purpose | Example Conditions |
| 0000 | 4 to 8 apheresis | Starting Material | Platelet Concentrates for |
| | platelet | | transfusion: Storage of not more |
| | concentrates for | | than 5 days at 25 ± 2° C. |
| | laboratory (pre- | | after donation. |
| | clinical) scale | | |
| | 100 to 150 | | |
| | apheresis | | |
| | platelet | | |
| | concentrates for | | |
| | engineering- & | | |
| | clinical batches | | |
| 0100 | Centrifugation | Separation of platelets | Centrifugation: 30 min at |
| | | (pellet) and plasma | 25 ± 2° C., at approximately |
| | | (supernatant) | 110 × g. |
| 0200 | Pellet | Resuspension of platelets | Addition of sterile saline to |
| | Resuspension in | in saline | replace the volume of the |
| | Saline 0.9% | | removed plasma. |
| 0300 | Freezing 1 | Induction of platelet | Freezing to below −30° C. |
| | | lysis; | |
| | | obtaining a storable and | |
| | | shippable intermediate | |
| 0400 | Intermediate | Storage below −30° C. | Storage/shipping conditions |
| | Product (Lysate): | | below −30° C. |
| 0500 | Thawing 1 | Platelet lysis | Complete thaw at a temperature |
| | | | of 30-37° C. for 30 ± 15 min. |
| | | | Homogenization of suspension |
| | | | by manual agitation. |
| 0600 | Freezing 2 | Platelet lysis | Freezing to obtain core |
| | | | temperature of less than −30° C., |
| | | | for at least 8 hours. |
| 0700 | Thawing 2 | Platelet lysis | Complete thaw at a temperature |
| | | | of 30-37° C. for 30 ± 15 min. |
| 0800 | Freezing 3 | Platelet lysis | Freezing to obtain core |
| | | | temperature of less than −30° C., |
| | | | for at least 8 hours. |
| 0900 | Thawing 3 | Platelet lysis | Complete thaw at a temperature |
| | | | of 30-37° C. for 30 ± 15 min. |
| 1000 | Centrifugation | Separation of cell debris | Centrifugation at 3000-4100 × g |
| | | and platelet lysate in the | at 2-8° C., for at least 30 min. |
| | | liquid phase | |
| 1100 | Pooling | Preparation of a | Transfer supernatant into a clean |
| | | homogenous pool | processing vessel at 2-8° C. |
| | | containing | Stirring of the solution to obtain a |
| | | approximately 1-2 | homogenous solution. |
| | | liters at laboratory | |
| | | scale (4-8 platelet | |
| | | concentrates) or | |
| | | approximately 20-30 | |
| | | liters of platelet lysate | |
| | | (100-150 platelet | |
| | | concentrates) for | |
| | | clinical scale | |
| 1200 | Platelet Pool | Starting material for | |
| | | down- stream | |
| | | processing | |
| 1300 | CaCl₂ Incubation | Removal of clottable | Addition of 1M CaCl₂ stock |
| | | protein | solution to obtain 23 mM CaCl₂. |
| | | | Incubation at 22 ± 3° C. for 75 to 90 |
| | | | min under gentle stirring. |
| 1400 | Centrifugation | Separation of clotted | Centrifugation at 22 ± 3° C. for at |
| | | protein (if any) and | least 30 min at 3000-4100 × g. |
| | | platelet lysate | Transfer supernatant through a |
| | | | 0.22 μm filter into a clean |
| | | | processing vessel. |
| 1500 | Heat Treatment | Virus Inactivation | Heat solution to 60.0 ± 1.0° C. and |
| | | | keep temperature for 180 min. |
| | | | Cool solution to 22 ± 3° C. |
| 1600 | Centrifugation | Separation of aggregates | Centrifugation at 22 ± 3° C. for at |
| | | (if any) and platelet | least 30 min at 3000-4100 × g. |
| | | lysate | |
| 1700 | Depth Filtration | Removal of aggregates | 3M Zeta-plus SP filter capsule |
| | | to prepare for 0.1 μm | (manufacturer: 3M) prerinsed |
| | | pre-Filtration. | with 50-60 L saline 0.9% per |
| | | | 1 m² of filter area. |
| | | | Approximately 25 cm² of filter |
| | | | area is required per 1 liter of |
| | | | platelet lysate. |
| | | | Filtration pressure at 2.5 bar or |
| | | | less at a temperature of 22 ± 3° C. |
| | | | Filter post wash: 4-10 liters |
| | | | water for injection per 1 m² of |
| | | | filter area. |
| | | | Mixing of lysate and post-wash |
| | | | to obtain a homogenous solution. |
| 1800 | 0.1 μm pre- | Removal of aggregates | Sterile Durapore 0.1 μm PVDF |
| | Filtration | prior to Nanofiltration | membrane (manufacturer: Merck |
| | | | Millipore) prerinsed with 10-15 |
| | | | liters saline 0.9% per 1 m² of |
| | | | filter area. Approximately 25 cm² |
| | | | per 1 liter of platelet lysate is |
| | | | required. Filtration pressure <2.5 |
| | | | bar. |
| 1900 | Nanofiltration 20 | Virus reduction | Planova S20N nanofilter |
| | nm | | nominal pore size 20 nm |
| | | | (manufacturer: Asahi Kasai), |
| | | | conditioned and integrity tested |
| | | | according to manufacturer's |
| | | | instructions. |
| | | | Filtration pressure of 2.2 bar or |
| | | | less. Temperature at 22 ± 3° C. |
| | | | Post wash: 5 to 15 liters of |
| | | | purified water per 1 m² of filter |
| | | | area. |
| | | | Nonfiltered lysate and post-wash |
| | | | are combined. |
| 2000 | Tangential Flow | Reduction of | Addition of a 25% human |
| | Filtration | process buffer salts, | albumin solution to obtain 2 g/l |
| | | i.e. NaCl and | human albumin in the |
| | | CaCl₂; | nanofiltrate. |
| | | Human albumin is added | Concentration to obtain |
| | | to the lysate ahead of the | approximately 25% of the |
| | | ultrafiltration to reduce | weight of the Platelet Pool [step# |
| | | the loss of growth fac- | 1200]. |
| | | tors. | Ultrafiltration against purified |
| | | | water (5 times the volume of the |
| | | | concentrated lysate solution). |
| | | | Temperature at 22 ± 3° C. |
| 2100 | Concentration | Achieve a concentration | Concentration to approximately |
| | | of the growth factor(s) | 20% the weight of the Platelet |
| | | sufficient to formulate | Pool [step# 1200], corresponding |
| | | the bulk solution | to 15-25% above the target |
| | | | concentration of TFG-β1 in the |
| | | | Formulated Bulk [step#2300] |
| 2200 | Bulk Formulation | Add stabilizers to | Bulk formulation: |
| | | achieve stability of the | Addition of human albumin |
| | | bulk solution prior to fill | solution 25 to 30 g albumin per |
| | | and finish | liter. |
| | | | Addition of pharmaceutical grade |
| | | | trehalose to 80 g per liter |
| | | | Adjust pH to 6.5-7.5, if |
| | | | necessary, by adding 0.1M |
| | | | NaOH or HCl. |
| | | | Dilute with water for injection |
| | | | to obtain target concentration |
| | | | of 300 ng of TGF-βl per 1 mL. |
| | | | Mixing to obtain a homogenous |
| | | | solution. |
| 2300 | Formulated Bulk | | |
| 2400 | 0.22 μm Filtration | Preparation for freezing | Transfer into a suitable single |
| | | of the formulated bulk | use bag. |
| | | solution | Pass through sterile 0.22 μm |
| | | | filter. |
| 2500 | Freezing <−30° C. | Obtain a storable | Freezing to obtain core |
| | | intermediate | temperature of below −30° C. |
| 2600 | Frozen Bulk | Extended storage | At or below −30° C. |
| 2700 | Thawing, | Preparation of sterile | Thawing to 22 ± 3° C. |
| | Homogenization | bulk solution | |
| 2800 | Sterile filtration | | Pass through sterile 0.22 μm |
| | | | PVDF filter into sterilized |
| | | | container. |
| 2900 | Sterile Bulk | Ready for aseptic filling | Immediate processing at |
| | Solution | | temperature of 22 ± 3° C. |
| 3000 | Aseptic Filling | Tube glass type I vials | Fill at temperature of 22 ± 3° C. |
| | | R20 bromo-butyl rubber | with 5.0 mL per vial. |
| | | stoppers or any suitable | Set rubber stopper half-way. |
| | | container | Transfer to freeze dryer under |
| | | | laminar flow. |
| 3100 | Freeze drying | Product stabilization | Cooling of shelf to 2 to 8° C. |
| | | | after secondary drying; hold |
| | | | shelf temperature until |
| | | | unloading. |
| | | | Closing of the vials under |
| | | | vacuum by pushing the stoppers |
| | | | to final position, fully sealing the |
| | | | vials. |
| 3200 | Capping | Secure container-closure | Firm pressing of aluminum |
| | | using a crimp cap | crimp cap with plastic flip-off as |
| | | | tampering evidence. |
| 3300 | Human Growth | Ready for product | Store at 2 to 8° C. |
| | Factor | testing and visual | |
| | Concentrate | inspection | |
| | (GFC- 01) | | |

### Example 11: In Vitro Cell-Based Assay to Measure Potency of Growth Factors

To measure potency and product quality, key soluble factors and responsive cell lines are identified and experiments performed to evaluate batch variability and/or dose response relationships, thereby resulting in a reliable bioassay to determine potency and quality.

To measure the relative potency of PLs, several factors are evaluated, including, but not limited to, batch or lot of PL, target cells exposed to factors, identification and relative and/or absolute amount of key soluble factors (includes cytokines, chemokines, growth factors, and others), and attributes of cells to be monitored as an indicator of activity (including metabolic activity; proliferation and cell division activities).

Different common cell lines known to show quantifiable responses to soluble factors of interest (e.g. TGF-β1) are evaluated and their relative proliferative response determined using methods known in the art. The cell lines being considered, with description of the cell type provided in parentheses, include: HepG2 (Epithelial-like hepatocarcinoma, human), HEK293 (Epithelial-like; Human Embryonic Kidney), CHO (Chinese Hamster Ovaries), C2C12 (Myoblast; targeted treatment can induce differentiation, mouse), THP-1 (Monocyte, Human), PC-12 (Neuronal from pheochromocytoma, rat), HeLa (Immortalized cervical carcinoma; human), MCF-7 (Adenocarcinoma—breast, human), NIH 3T3 (Fibroblast; mouse), and hTERT (Fibroblast, human).

To assess batch variability, 3 to 5 batches are used, along with possible combinations thereof, to test for special effects and additivity. Each batch is applied in serial dilution to create a dose-response curve (linear or asymptotic). Replicates of each batch are tested in each of the targeted cell lines. Responses (i.e. relative potency) are measured versus either a designated “reference batch” (arbitrary) and/or against a batch created by combination of positive controls (soluble factors) at known concentrations.

For comparison and to confirm the general applicability of the model systems, commercially available factors known to elicit proliferative responses and, ideally, known to be present in PL, are used as positive controls and to identify reasonable conditions for the assays.

The viability and proliferation of cells are determined using a broad range of methodologies. Some common assays used to determine cell viability or proliferation include, but are not limited to, Alamar Blue Misc, CellTiter-Glo Promega, TACSXTT RND Systems, MTT Cell Proliferation RND Systems, Calcein AM RND Systems, Propidium Iodide Misc, Annexin V Misc, and BrdU DNA synthesis.

To test proliferative response to human platelet-derived growth factor concentrate, cells are exposed to varying concentrations of the factors with and without control soluble factors and compared to negative controls (media; buffer). To evaluation dose response, cells are exposed to controlled dilutions of the solution with secreted factors across a controlled range with the intent of observing a response that is a 4PL or linear response to the concentration (dilution factor) of sample.

### Example 12: Cell Culture and In Vitro Studies

Proliferation assays were conducted to investigate the effect of human platelet-derived growth factor concentrate (GFC-01) on proliferation of human skin fibroblasts and epidermal keratinocytes and rat skin fibroblasts and epidermal keratinocytes. Cells cultured in fibroblast and keratinocyte growth medium supplemented with 10% fetal bovine serum and 1% penicillin-streptomycin until passage 4 were exposed to various concentrations of GFC-01, human recombinant EGF and TGF-0, and rat recombinant EGF and TGF-0 as control.

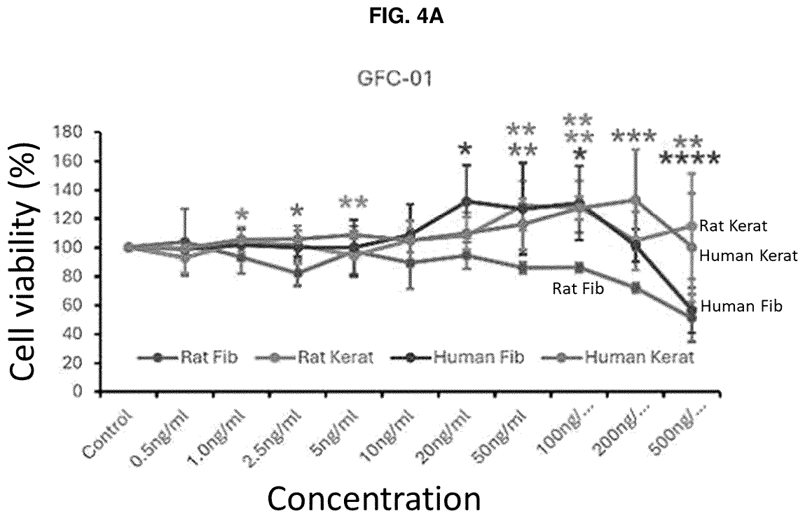

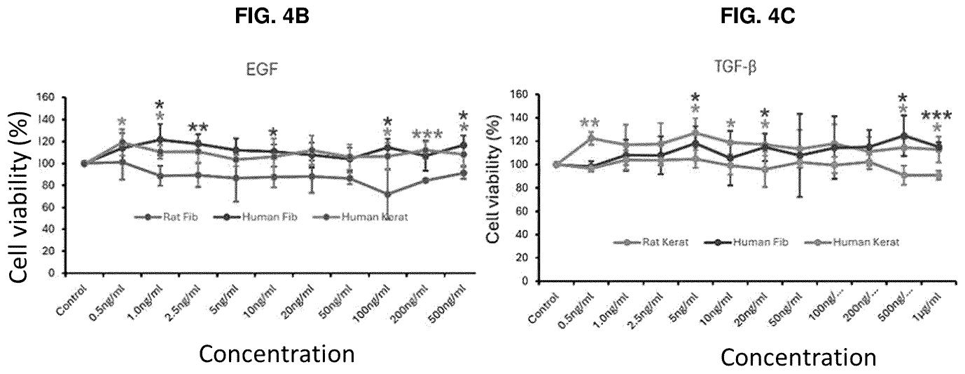

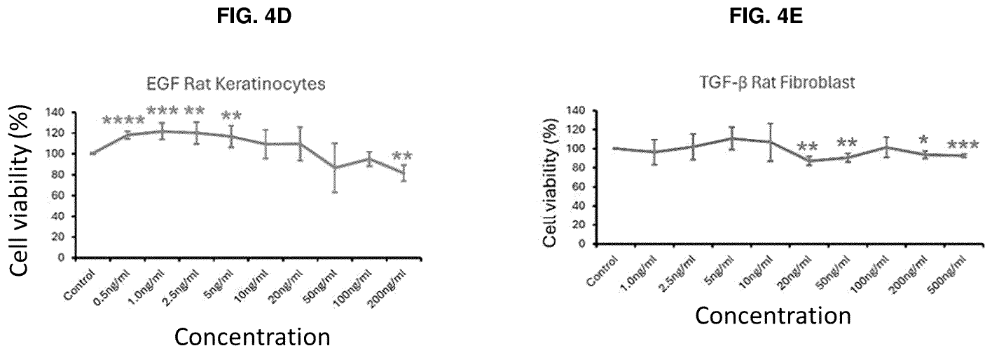

Twenty-four hours after treatment with GFC-01, 3-(4,5-dimethylthiazol-2-yl)-2,5-diphenyltetrazolium bromide assay (MTT assay) was carried out and read using Agilent BioTek Gen 5 plate reader. As shown in FIG. 4A, results from the proliferation assays suggest that: (1) GFC-01 does not affect human fibroblast proliferation up to at least a concentration of 200 ng/ml (all concentrations are based on TGF-0 concentration in GFC-01), (2) GFC-01 can promote human fibroblast proliferation between concentrations of 10-100 ng/ml, (3) GFC-01 does not affect human keratinocytes proliferation up to at least a concentration of 500 ng/ml and can promote human keratinocyte proliferation in a concentration-dependent manner between the concentrations of 1-22 ng/ml, (4) GFC-01 does not affect proliferation of rat fibroblasts and keratinocytes up to at least a concentration of 200 ng/ml, and (5) GFC-01 does not affect the proliferation of rat keratinocytes up to at least a concentration of 500 ng/ml and promotes rat keratinocyte proliferation in a concentration-dependent manner between the concentrations of 5-100 ng/ml. FIGS. 4B-4E show that human recombinant EGF and TGF-3 and rat recombinant EGF and TGF-β used as controls promoted proliferation of respective cell lines as expected.

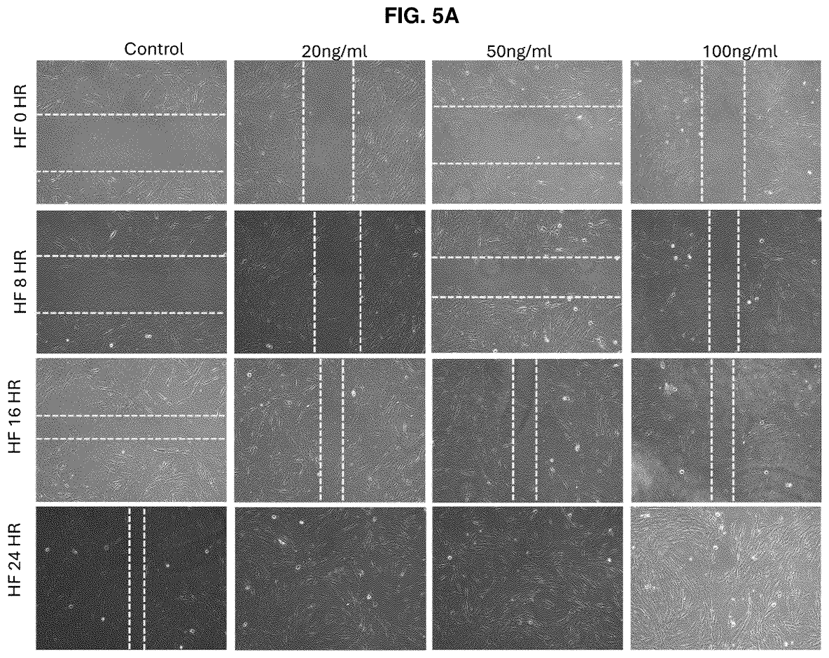

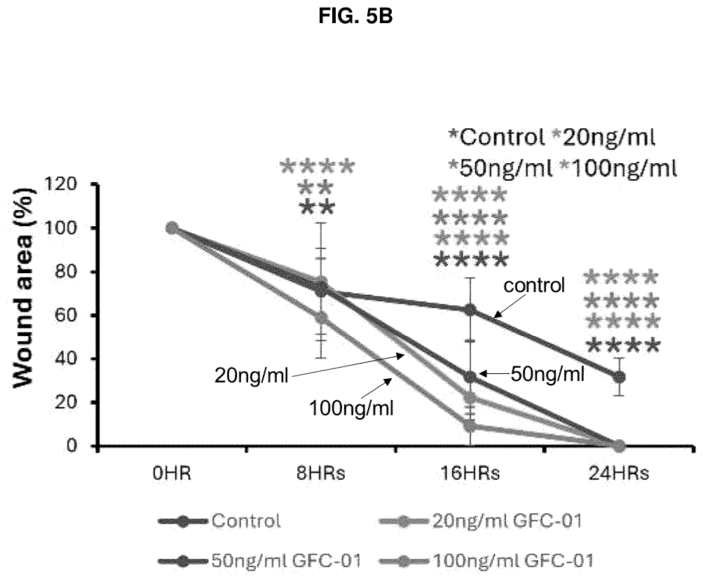

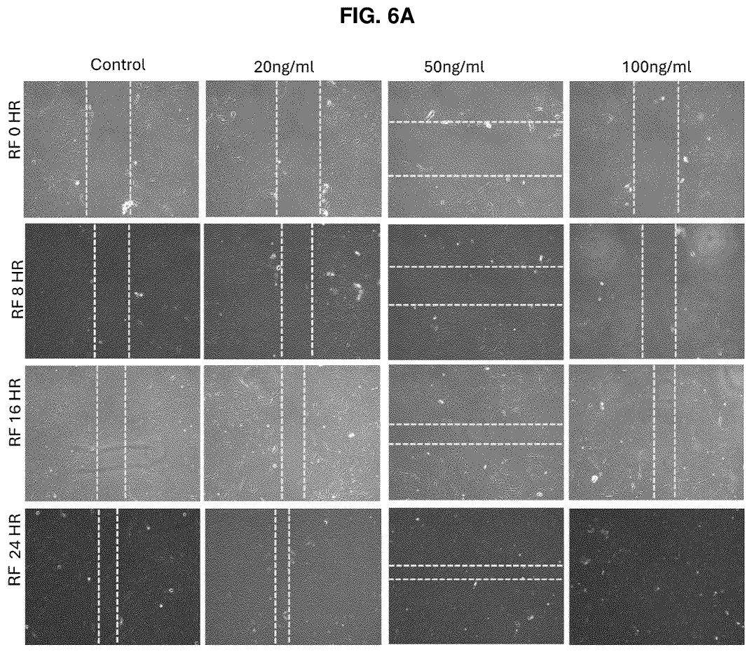

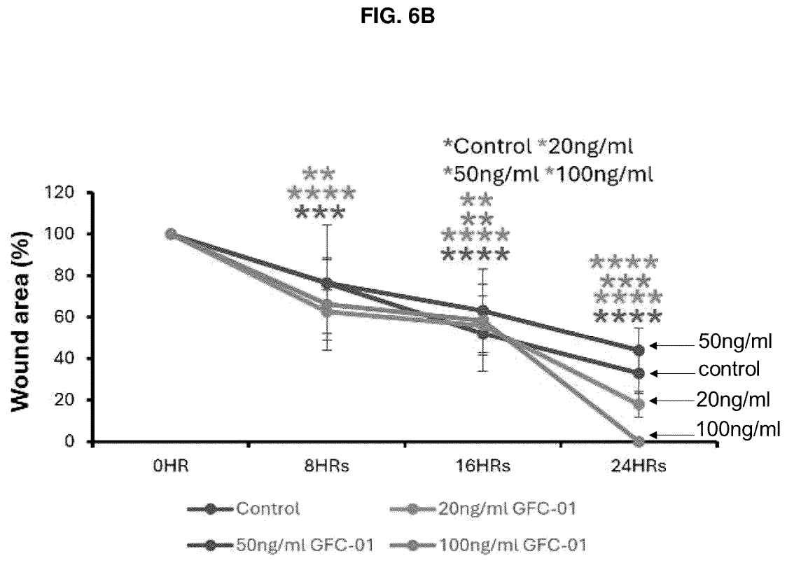

Migration assays were carried out using a wound healing assay in a 6-well plate scanned at multiple time points using human growth factor concentrate (GFC-01), human recombinant EGF and TGF-0, and rat recombinant EGF and TGF-0 as control. All experiments were carried out in triplicates with a minimum of three biological replicates. Results from the migration assays assay suggest a concentration and time-dependent increase in migration of human fibroblasts in the presence of GFC-01, as shown in FIGS. 5A and 5B. Rat fibroblasts also showed a concentration and time-dependent increase in migration with GFC-01 as shown in FIGS. 6A and 6B.

### Example 13: Effects of Human Growth Factor Concentrate on Wound Healing

Sprague Dawley rats (n=70), both male and female, aged 6-8 weeks and weighing approximately 175 g, were used to test the efficacy and dosing of a biologic human growth factor concentrate (GFC-01) in promoting wound healing using a diabetic rat model. The animals were obtained from Charles River Laboratories (USA) and housed in the Western University of Health Sciences, Pomona, CA Animal Resource Facility under standardized conditions, including a constant temperature of 22° C. and a 12-hour light/dark cycle, following the facility's Standard Operating Procedures.

Power analysis was performed with consideration of the primary outcome of animal weight and the secondary outcome of blood sugar levels. The sample size necessary to have at least 95% power to detect significant change was determined to be 10 in each group. A minimum of 14 rats was used in each group. The 70 rats were randomly divided into two groups: a control nondiabetic group (n=14) and a diabetic group (n=56; n=14 each in 0.5 ml, 1.0 ml, and 1.5 ml GFC-01). Animals in the control group were fed a normal diet (ND; 20% protein, 70% carbohydrate, 10% fat; D12450B; Research Diet Inc.) and water ad libitum. Animals in the diabetic groups received a high-fat diet (HFD; 35% carbohydrate, 20% protein, 45% fat; 5.7 kcal/g total; D12451; Research Diet Inc.) and water available ad libitum. The rats were weighed three times: initially, at the start of the HFD; after 6 weeks of the HFD, before diabetes induction; and after 9 weeks of the HFD, following diabetes development. Blood sugar was also measured initially at the start, before inducing diabetes, two weeks after streptozotocin (STZ) injection, and at the end of the experiment using tail vein blood.

To induce type II diabetes mellitus (T2D), both male and female rats in the diabetic group were injected intraperitoneally (IP) with a low dose of streptozotocin (STZ; 25 mg/kg, dissolved in 0.1M sodium citrate buffer, pH 4.4; Sigma-Aldrich, St. Louis, MO) after 6 weeks of HFD. To induce T2D in ND rats (n=20, 10 male and 10 female), two STZ injections were administered, followed by a second dose 7 days after the first STZ injection. Rats in the control group were injected with vehicle citrate buffer (0.25 ml/kg) at the same point. Hyperglycemia typically developed consistently in diabetic rats around 2 weeks post-induction.

Blood glucose levels were monitored in both groups using tail vein blood samples and measured with an AlphaTrak glucometer. Rats with a blood glucose level exceeding 250 mg/dl after 2 weeks were considered diabetic. To induce ulcers, rats were anesthetized, the dorsal surface fur was shaved using an electric hair clipper, and the skin was cleaned with a 70% ethanol wipe. A standardized full-thickness open excision wound was created, one on both sides of the upper spine (equidistant from the thoracic spine), using a sterile biopsy punch (Cat no. 12-460-412, Thermo Fischer Scientific, USA) with a 6 mm in diameter and 2 mm in depth. After wound creation, the rats were allowed to recover on a heating pad (64° F. to 79° F.). All animals were continuously monitored in the immediate postoperative period until the rats were awake and started moving. Rats were kept back in their cage and monitored daily. To prevent wound infection after wound induction, one dose of antibiotic was given to the rats. To attenuate pain, buprenorphine was given subcutaneously.

The rats were used to evaluate the progression of wound healing and to assess the effects of GFC-01 given to rats subcutaneously around the wound in different doses on day 0 and day 3. Three doses of GFC-01 were tested: 0.5 ml dose containing 150 ng of growth factors, 1.0 ml dose containing 300 ng of growth factors, and 1.5 ml dose containing 450 ng of growth factors. All ulcers were photographed at indicated time points using a camera to evaluate the ulcer healing progress.

In-vivo results: Male rats developed diabetes after 2 STZ injections while most of the female rats developed diabetes after 3 STZ injections. There was no correlation between the weight and development of diabetes. Average wound area as a percentage of the wound area as measured on Day 0 was calculated for Days 3, 7, 10, 14, and 21 and are presented in Table 2 below. Additional days on which wound area was determined are indicated in Table 2.

TABLE 2
Average wound areas

| | Average wound area as a percentage of wound area | | | | | |
|---|---|---|---|---|---|---|
| | measured on Day 0 | | | | | |
| | Day 0 | Day 3 | Day 7 | Day 10 | Day 14 | Day 21 |
| Control | 100 | 63.8 | 12 | 3.6 | 0 | — |
| Diabetic | 100 | 92 | 42 | 16 (D12) | 10 (D15) | 0 |
| Diabetic 1 ml GFC-01 | 100 | 62 | 16 | — | 0 (D13) | — |
| Diabetic 0.5 ml GFC-01 | 100 | 88 | 34 | 9 | 0 | — |
| Diabetic 1.5 ml GFC-01 | 100 | 80 | 35 | 9 | 0 | — |

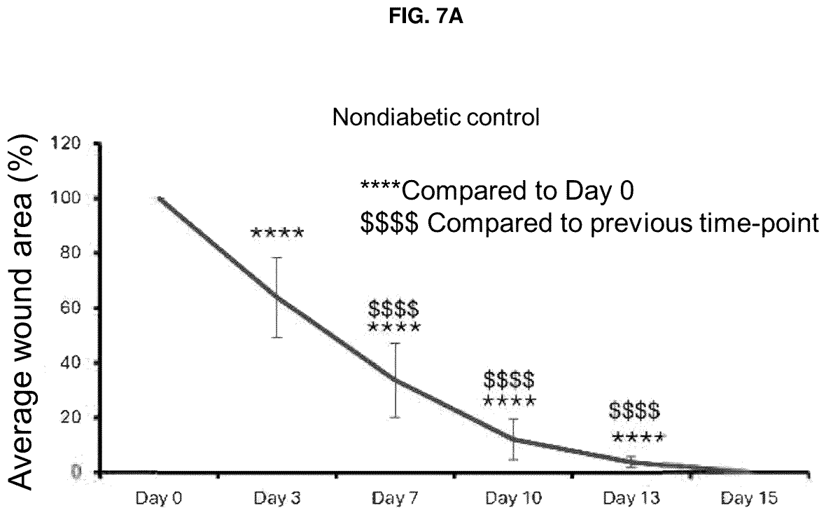

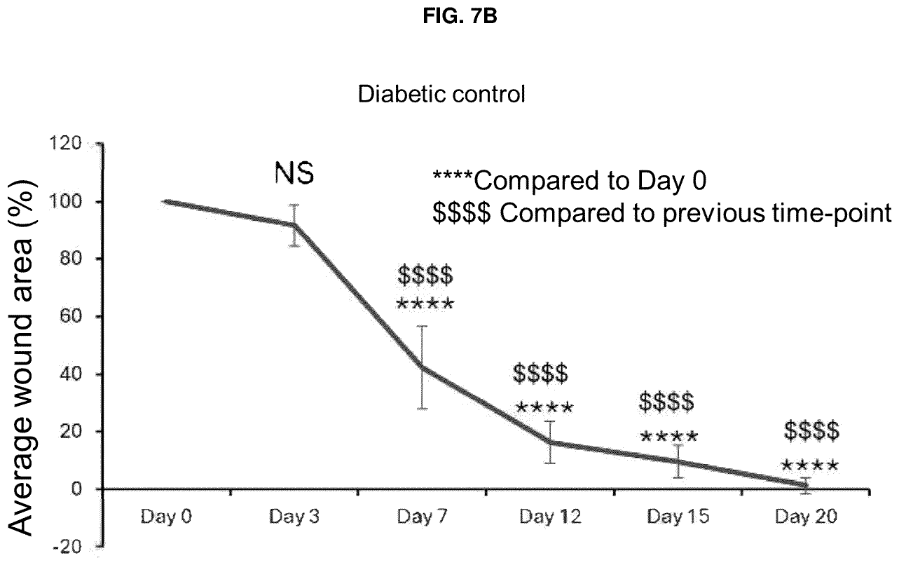

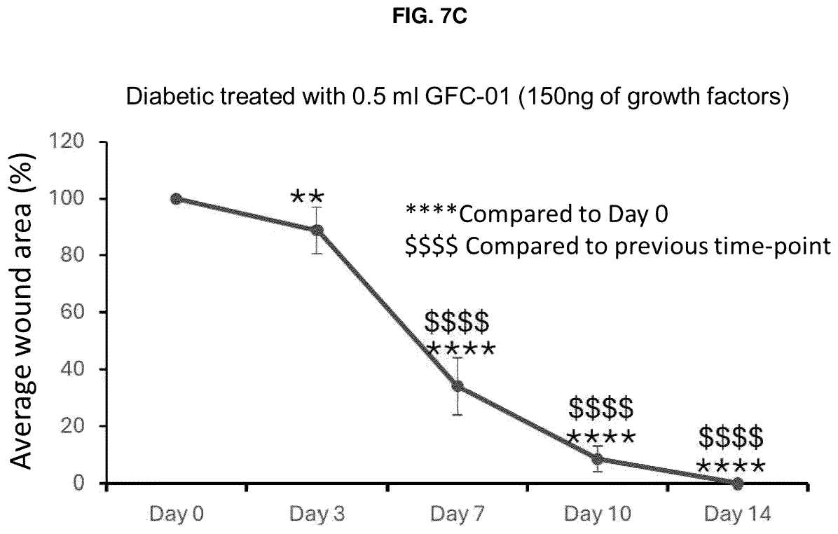

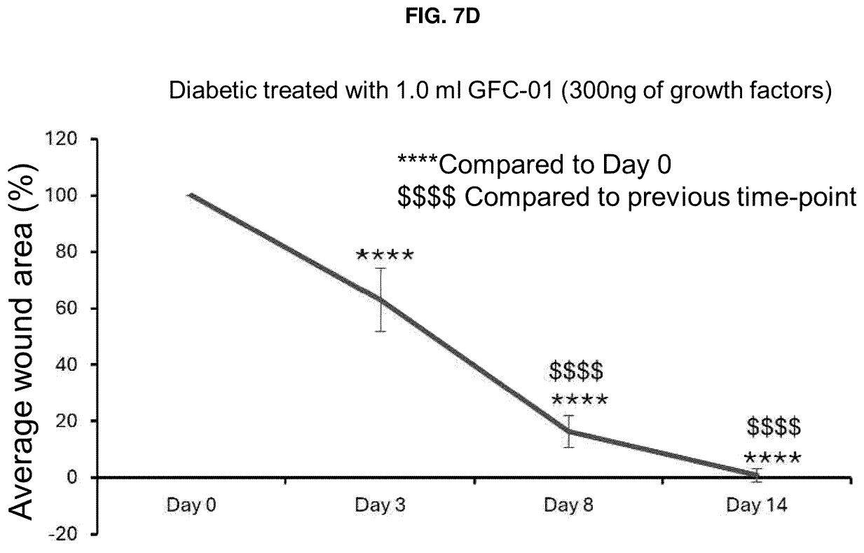

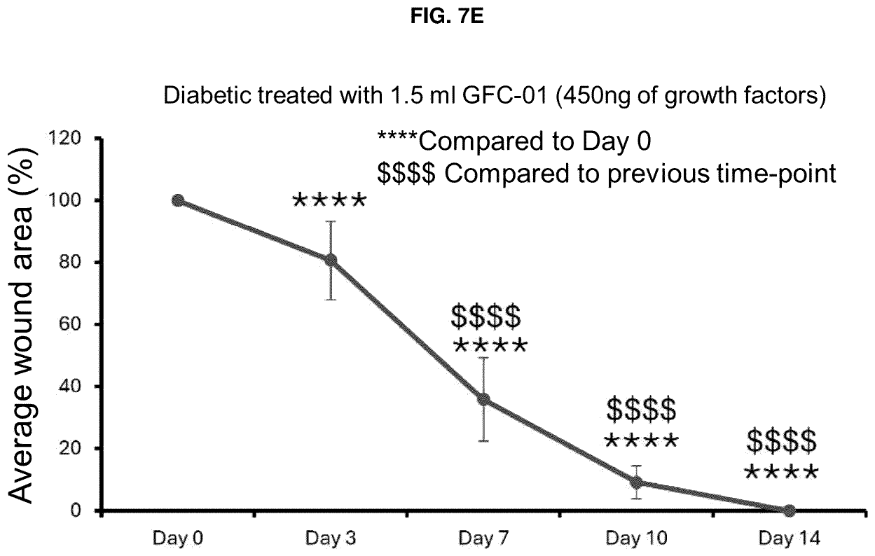

As shown in FIG. 7A, wounds healed in 14 days in nondiabetic nontreated control rats. As shown in FIG. 7B, wound healed in 21 days in diabetic nontreated rats. As shown in FIGS. 7C-7E, GFC-01 at doses of 0.5 ml, 1.0 ml, and 1.5 ml promoted wound healing in diabetic rats, with wounds healing by D13/14. The fastest decrease in wound area was seen with administration of 1 ml GFC-01 at D3 and D7.

Tissues from the rats in all treatment groups were collected. Molecular and cellular studies, including histology, immunostaining, PCR, Western-Blot analysis, are performed to analyze the collected tissues.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A method for preparing a therapeutic composition comprising one or more human platelet derived growth factors, comprising of:
a. collecting a supernatant from a separated platelet lysate to obtain a solution comprising growth factors; optionally filtering the supernatant to obtain a clear solution;
b. inactivating viruses in the solution of step (a) by a first virus elimination step; and
c. generating a filtrate by passing the solution from step (b) through a nanofilter as a second virus elimination step.

2. The method of claim 1, further comprising concentrating the filtrate from step (c) to obtain a suitable concentration of the one or more human platelet derived growth factors.

3. A method for preparing a therapeutic composition comprising one or more human platelet derived growth factors, comprising of:
a. providing platelet lysates from at least two donors;
b. diluting the platelet lysates with a serum-free diluent to create a mixture;
c. separating the mixture into a supernatant and a pellet;
d. collecting the supernatant to obtain a solution comprising growth factors; optionally filtering the supernatant to obtain a clear solution;
e. inactivating viruses in the solution of step (d) by a first virus elimination step;
f. generating a filtrate by passing the solution from step (e) through a nanofilter as a second virus elimination step; and
g. concentrating the filtrate from step (f) to obtain a suitable concentration of the one or more human platelet derived growth factors.

4. The method of claim 3, wherein the platelet lysates are previously frozen.

5. The method of claim 3 or 4, wherein the platelet lysates from step (a) are obtained from human platelet concentrates, buffy coats, or platelet rich plasma, wherein obtaining the platelet lysates comprises lysis of at least a portion of the platelets in the platelet concentrates, buffy coats, or platelet rich plasma.

6. The method of claim 5, wherein the platelet concentrates, buffy coats, or platelet rich plasma are negative for the presence of pathogens.

7. The method of claim 5 or claim 6, wherein, prior to lysis of the platelets, at least a portion of plasma is removed from the platelets and a serum-free solution is added.

8. The method of any one of claims 5-7, wherein obtaining the platelet lysates comprises:
a. separating the human platelet concentrates or platelet rich plasma into a supernatant and a pellet containing the platelets;
b. resuspending the pellet containing the platelets into a solution, wherein the solution is serum-free and/or comprises human albumin;
c. freezing the resuspended platelets; and
d. thawing the resuspended platelets.

9. The method of claim 8, wherein steps (c), freezing the resuspended platelets, and step (d), thawing the resuspended platelets, are repeated for one or more times.

10. The method of claim 8 or claim 9, wherein the resuspended platelets reach a core temperature of at least −18° C. after freezing.

11. The method of claim 10, wherein after freezing, the platelets are stored at a temperature of at least −18° C., preferably below −30° C.

12. The method of any one of claims 8-11, wherein the platelets are stored for a period of up to 12 months after freezing.

13. The method of any one of claims 3-12, wherein the serum-free diluent is a calcium chloride solution.

14. The method of claim 13, wherein the concentration of the calcium chloride is 15-25 mM.

15. The method of claim 14, wherein the concentration of the calcium chloride is 20 mM.

16. The method of any one of claims 1-15, wherein one or more detergents and/or solvents are added to the mixture prior to dilution of the platelet lysates with the serum-free diluent.

17. The method of claim 16, wherein the one or more detergents comprises a non-ionic detergent.

18. The method of claim 16 or claim 17, wherein the one or more detergents comprises a polysorbate derivative, a nonylphenol derivative, a cholic acid derivative, or a combination thereof.

19. The method of claim 18, wherein the one or more detergents comprises a polysorbate derivative.

20. The method of claim 19, wherein the polysorbate derivative is polysorbate 80 (Tween 80).

21. The method of claim 18, wherein the one or more detergents comprises a nonylphenol derivative.

22. The method of claim 21, wherein the nonylphenol derivative is Triton-X-100.

23. The method of claim 18, wherein the one or more detergents comprises a cholic acid derivative.

24. The method of claim 23, wherein the cholic acid derivative is sodium cholate.

25. The method of any one of claims 16-24, wherein the concentration of the detergent in the mixture is 1%.

26. The method of any one of claims 16-25, wherein the one or more solvents comprises tri-n-butyl phosphate.

27. The method of any one of claim 16-26, wherein the concentration of the solvent in the mixture is 0.3%.

28. The method of any one of claims 1-27, wherein the first virus elimination step comprises heating the solution, lowering the pH of the solution, exposing the solution to UV light, ultrafiltration of the solution, or adding solvent/detergent reagents to the solution.

29. The method of claim 28, wherein the first virus elimination step comprises exposing the solution to UV light.

30. The method of claim 28, wherein the first virus elimination step comprises heating the solution.

31. The method of claim 30, wherein the solution is heated to about 60 degrees Celsius.

32. The method of claim 31, wherein the solution is heated to said temperature for at least two hours.

33. The method of claim 28, wherein the first virus elimination step comprises lowering the pH of the solution.

34. The method of claim 33, wherein the pH of the solution is lowered to about 4.0.

35. The method of claim 34, wherein the solution is incubated at said pH for up to 10 hours.

36. The method of claim 35, wherein the solution is incubated at about 37 degrees Celsius.

37. The method of claim 28, wherein the first virus elimination step comprises adding solvent/detergent reagents to the solution.

38. The method of claim 37, wherein the solvent/detergent reagents comprise a non-ionic detergent.

39. The method of claim 37 or claim 38, wherein the solvent/detergent reagents comprise a polysorbate derivative, a nonylphenol derivative, a cholic acid derivative, or a combination thereof.

40. The method of claim 39, wherein the solvent/detergent reagents comprise a polysorbate derivative.

41. The method of claim 40, wherein the polysorbate derivative is polysorbate 80 (Tween 80).

42. The method of claim 39, wherein the solvent/detergent reagents comprise a nonylphenol derivative.

43. The method of claim 42, wherein the nonylphenol derivative is Triton-X-100.

44. The method of any one of claims 37-43, wherein the concentration of the detergent in the solution is 1%.

45. The method of any one of claims 37-44, wherein the one or more solvents comprises tri-n-butyl phosphate.

46. The method of any one of claim 37-45, wherein the concentration of the solvent is 0.3%.

47. The method of any one of claims 1-46, wherein the nanofilter has a pore size of 15-25 nm.

48. The method of any one of claims 2-47, wherein concentrating the filtrate comprises ultrafiltration.

49. The method of claim 48, wherein the ultrafiltration is tangential flow filtration.

50. The method of claim 49, wherein the tangential flow filtration is done against 3 to 5 volumes of purified water or a buffer suitable for injection.

51. The method of claim 50, wherein the buffer is saline.

52. A therapeutic composition made by the method of any one of claims 1-51.

53. A pharmaceutical composition comprising the composition of claim 52 and a pharmaceutically acceptable excipient.

54. The pharmaceutical composition of claim 53, comprising one or more human platelet derived growth factors selected from the group consisting of: platelet derived growth factor BB (PDGF-BB), epidermal growth factor (EGF), vascular endothelial growth factor (VEGF), nerve growth factor beta (NGFO), Transforming growth factor beta 1 (TGF-β1), platelet derived growth factor AB (PDGF-AB), and insulin-like growth factor 1 (IGF-1).

55. The pharmaceutical composition of claim 54, wherein the concentration of the one or more human platelet derived growth factors is between 1 and 1000 ng/mL.

56. The pharmaceutical composition of any one of claims 53-55, comprising one or more stabilizers selected from the group consisting of human albumin, sucrose, trehalose, maltose, sorbitol, and hydroxyethyl-starch.

57. The pharmaceutical composition of claim 56, comprising 10% sucrose or trehalose.

58. The pharmaceutical composition of any one of claims 53-57, comprising one or more amino acids selected from the group consisting of L-arginine, L-lysine, and N-acetyltryptophan.

59. The pharmaceutical composition of claim 58, wherein the concentration of the amino acids is 250 mmol/l.

60. The pharmaceutical composition of any one of claims 53-59, further comprising between 10 and 100 mg/mL of human albumin.

61. The pharmaceutical composition of any one of claims 53-60, comprising a buffering agent selected from the group consisting of sodium citrate, sodium phosphate, and sodium acetate.

62. The pharmaceutical composition of claim 61, wherein the buffering agent maintains a pH of between 6.0 and 8.5, preferably wherein the pH is 7.2.

63. The pharmaceutical composition of any one of claims 53-62, wherein the osmolality is 350 mOsmol/kg or less.

64. The pharmaceutical composition of claim 63, wherein the osmolality is 300 mOsmol/kg.

65. The pharmaceutical composition of any one of claims 53-64, wherein the composition is lyophilized.

66. The pharmaceutical composition of any one of claims 53-65, wherein the composition is endotoxin free.

67. The pharmaceutical composition of any one of claims 53-66, wherein the composition is free of viruses.

68. The pharmaceutical composition of claim 67, wherein the composition is free of bloodborne non-enveloped and enveloped viruses.

69. The pharmaceutical composition of any one of claims 53-68, wherein the pharmaceutical composition is safe for subcutaneous injection, intradermal injection, transdermal injection, subdermal injection, intramuscular injection, and intraarticular injection.

70. A method of treating a condition or disease in a subject in need thereof, comprising administering to the subject the pharmaceutical composition of any one of claims 53-69.

71. The method of claim 70, wherein the pharmaceutical composition is administered through subcutaneous injection, intradermal injection, transdermal injection, subdermal injection, intramuscular injection, intraarticular injection, topical application, or a combination thereof.

72. The method of claim 71, wherein the pharmaceutical composition is administered through subcutaneous injection, intradermal injection, transdermal injection, or subdermal injection.

73. The method of any one of claims 70-72, wherein the condition or disease is selected from the group consisting of non-healing wounds or ulcers, chronic and acute dermatological disorders, intra-abdominal abscesses, neurodegenerative diseases, muscle injuries, tendonitis, ligament injuries, and osteoarthritis.

74. The method of claim 73, wherein non-healing wound or ulcer is selected from the group consisting of diabetic foot ulcers, corneal ulcers, acute wounds, burns, acute external surgical wounds to the epidermis, acute surgical wounds to an internal organ, traumatic wounds, and atraumatic wounds.

75. The method of claim 74, wherein the chronic and acute dermatological disorder is eczema or alopecia.
